(19) United States
(12) Patent Application Publication
(10) Pub. No.: US 2012/0264190 A1
(43) Pub. Date: Oct. 18, 2012

(54) Decellularized and Delipidized Extracellular Matrix and Methods of Use

(75) Inventors: Karen L. Christman, San Diego, CA (US); D. Adam Young, San Diego, CA (US)

(73) Assignee: THE REGENTS OF THE UNIVERSITY OF CALIFORNIA, Oakland, CA (US)

(21) Appl. No.: 13/489,567

(22) Filed: Jun. 6, 2012

Related U.S. Application Data
(63) Continuation of application No. PCT/US2010/061436, filed on Dec. 21, 2010.
(60) Provisional application No. 61/288,402, filed on Dec. 21, 2009.

(51) Int. Cl. C12N 5/07; C12N 9/50

(52) U.S. Cl. 435/219; 435/404; 435/405; 435/395

(57) ABSTRACT

Compositions comprising decellularized and delipidized extracellular matrix derived from adipose or loose connective tissue, and therapeutic uses thereof. Methods for treating, repairing or regenerating defective, diseased, or damaged adipose or loose connective tissues or organs in a subject, preferably a human, and/or for tissue engineering, filing soft tissue defects, and cosmetic and reconstructive surgery, using a decellularized and delipidized adipose or loose connective tissue extracellular matrix of the invention are provided. Methods of preparing tissue culture surfaces and culturing cells with adsorbed decellularized and delipidized adipose or loose connective tissue extracellular matrix are also provided.

### CROSS REFERENCES TO RELATED APPLICATIONS

This patent application is a continuation of PCT Application No. PCT/US2010/061,436, filed Dec. 21, 2010, which claims priority benefit of U.S. Provisional Application No. 61/288,402, filed Dec. 21, 2009, each of which is incorporated herein by reference in their entireties.

### STATEMENT OF GOVERNMENT INTEREST

This invention was made with government support under grant No. 1DP20D004309-01 awarded by National Institutes of Health (NIH). The government has certain rights in the invention.

### BACKGROUND

Adequate replacement of adipose tissue is often overlooked when restructuring soft tissues for aesthetic improvement or traumatic injury repair. In addition to its roles in energy storage and cushioning, adipose tissue also significantly contributes to bodily symmetry and aesthetics. Several researchers have investigated traditional biomaterials for adipogenic capability, but each one faces significant drawbacks, as it was not originally tailored for adipose tissue. Common synthetic polymers, such as poly(lactic-co-glycolic acid) (PLGA), have proven insufficient to cause natural regeneration of adipocytes and face some degree of fibrous encapsulation in animal models [1]. Natural biopolymers, such as collagen and hyaluronic acid, have also been molded into gels and cross-linked scaffolds. These materials improve biocompatibility but struggle to resist rapid resorption [2,3]. Clinical trials of hyaluronic acid scaffolds have shown maintained shape and cellular infiltration, but the implants suffered from limited integration and an absence of mature adipocytes within the material [3].

In addition to an inability to adequately induce adipogenesis, these three dimensional scaffolds also require surgical implantation. To minimize the invasive delivery of materials for adipose regeneration, several natural and synthetic polymers with injectable functionality have been investigated for in vivo adipogenic potential. Alginate and fibrin have been extensively studied because they readily gel and their biocompatibility is well known [4,5]. These studies have shown positive cell survival and improved vascularization following implantation. However, acellular implants exhibited limited formation of new adipose tissue, and the presence of foreign body giant cells and a fibrous capsule [4,6]. Recently, collagen and hyaluronic acid have emerged as popular soft tissue fillers and are the major components of several commercially available products. Collagen has a low incidence of allergic reaction but, in an injectable form, can be rapidly resorbed and encourages only limited adipogenesis [7,8]. Hyaluronic acid has shown improved angiogenesis and adipogenesis; however, it too faces rapid resorption in vivo [9, 10]. Tan et al. recently introduced a modified version of hyaluronic acid linked to poly-(N-isopropylacrylamide) that self-assembles at body temperature, but it has yet to be tested for adipogenic potential [1,1]. Despite the availability of several injectable materials, there has yet to be identified an engineered material that avoids immune complications and encourages new fat formation. Moreover, no injectable material has been designed to mimic the native adipose extracellular matrix (ECM).

Several clinicians have pursued autologous alternatives by using free fat transfer to augment soft tissues [12, 13]. These “lipotransfer” treatments inject liposuctioned fat back into a patient through a cannula inserted into the subcutaneous space. This process has seen initial short-term success in small volume areas and a limited immune response [1,4]. However, mature adipocytes are poorly equipped to survive ischemic conditions which leads to rapid necrosis and resorption in many cases [1,5]. The lipoaspirate also exhibits variable mechanical properties and requires an 18 G needle to accommodate the viscous emulsion of adipose particulate [1,6]. Lipotransfer provides a material that contains many of the natural components of adipose tissue and consequently has promoted adequate integration with host tissue. However, the inability to control the composition or mechanics of lipoaspirate results in unpredictable implant contours and resorption.

Decellularization of tissues has recently emerged as a major player in the field of regenerative medicine and offers the possibility of producing a scaffold that closely mimics the physical and chemical cues seen by cells in vivo [17, 18]. Materials produced in this manner often have positive angiogenic and chemoattractant properties [19-22]. A couple tissues have been decellularized for use in adipose regeneration studies with promising results, including skeletal muscle and placental tissue [23, 24]. However, these scaffolds do not directly match the composition of the native adipose ECM. While many tissues share similar ECM elements, it is becoming evident that each tissue has its own complex composition and concentration of chemical constituents [25], which are known to regulate numerous cell processes including attachment, survival, migration, proliferation, and differentiation [26-31]. It follows that the use of decellularized adipose tissue would provide the best matrix for adipose regeneration.

Recently, a couple of groups have investigated the potential to generate an acellular material from human adipose tissue [32, 33]. While successful in removing a majority of the cellular content, these methods resulted in three-dimensional scaffolds. These products would necessitate surgical implantation and limit customization for varying dimensions in the subcutaneous space.

Thus, there exists a need for an acellular, injectable material that will satisfy complex contours while also closely mimicking the complexity of natural adipose ECM. Processing of adipose ECM removed via liposuction could eliminate the necrosis and variability associated with current lipotransfer procedures. Further, there exists a need for improved compositions for adipose tissue repair, regeneration, and adipocytes or lipoblasts cell culture. Similarly, there also exists a need for improved compositions for loose connective tissue repair, regeneration and cell culturing.

### SUMMARY OF THE INVENTION

The present invention provides a composition comprising a decellularized and delipidized extracellular matrix and method of use thereof. More particularly, the present invention provides that the decellularized and delipidized extracellular matrix of the present invention is derived from adipose or loose connective tissue. In certain embodiments, the decellularized and delipidized adipose matrix of the present invention is derived from the lipoaspirate obtained from liposuction of the adipose or loose connective tissue, and comprises native glycosaminoglycans, proteins or peptides.

In one aspect, the invention provides a composition comprising decellularized and delipidized extracellular matrix derived from adipose or loose connective tissue for adipose tissue engineering, filling soft tissue defects, and cosmetic and reconstructive surgery. In some instances, the adipose tissue or body fat or just fat is loose connective tissue composed of adipocytes. Fat in its solitary state exists in the liver, heart, and muscles. Loose connective tissue includes areolar tissue, reticular tissue and adipose tissue. Adipose tissue is derived from adipocytes and/or lipoblasts.

The composition of the present invention can be injectable, and formulated to be in liquid form at room temperature, typically 20° C. to 25° C., and in gel form at a temperature greater than room temperature, e.g., 25° C., or at normal body temperature, e.g., 37° C. Therefore, in certain embodiments, the composition of the present invention is a thermally responsive hydrogel that is in a liquid form at room temperature and in gel form at a temperature greater than room temperature or at normal body temperature.

In some instances, the adipose tissue comprises white adipose tissue (WAT) or brown adipose tissue (BAT), and is selected from the group consisting of human adipose tissue, primate adipose tissue, porcine adipose tissue, bovine adipose tissue, or any other mammalian or animal adipose tissue, including but not limited to, goat adipose tissue, mouse adipose tissue, rat adipose tissue, rabbit adipose tissue, and chicken adipose tissue.

In some instances, the composition is configured to be injected into a subject in need at a desired site for tissue engineering, filling soft tissue defects or cosmetic or reconstructive surgery. In some instances, the composition is configured to be delivered to a tissue through a small gauge needle (e.g., 25 gauge or smaller). In some instances, the composition of the present invention can be gelled, modified and manipulated into a desired shape in vivo after injection. In one aspect of the present invention, the composition can be injected in particulate form or digested to create a solution that self-assembles into a gel after injection into the site. In some instances, the composition of the present invention can be gelled, modified and manipulated into a desired form ex vivo and then implanted. In some instances, the composition of the present invention can be crosslinked with a molecule, such as glutaraldehyde, 1-ethyl-3-[3-dimethylaminopropyl]carbodiimide hydrochloride (EDC) or transglutaminase, to increase material stiffness and modulate degradation of the composition.

In some instances, the composition comprises naturally or non-naturally occurring chemotaxis, growth and stimulatory factors that recruit cells into the composition in vivo. In some instances, the composition further comprises a population of exogenous therapeutic agents to promote repair or regeneration. In some instances, the composition of the present invention is configured as a delivery vehicle for therapeutic agents, cells, proteins, or other biological materials. In one embodiment, the composition of the present invention can be used to deliver platelet-rich plasma (PRP) that is derived from whole blood of the patient or from another blood donor. The cells that can be delivered by the composition of the present invention include, but are not limited to, pluripotent or multipotent stem cells, mesoderm precursor cells, adipocytes, lipoblasts, or precursors thereof, e.g., human adipose derived stem cells, progenitor cells, adipose-derived mesenchymal stem cell, other adipose tissue-related cells, or any other derived or induced stem or progenitor cells from other tissues.

The composition comprising the decellularized and delipidized adipose extracellular matrix of the present invention can also be used as a substrate to culture adipose- and/or other tissue-derived stem cells. In some instances, the composition is configured to coat surfaces, such as tissue culture plates or scaffolds, to culture adipocytes and lipoblasts or other cell types, such as adipose-derived mesenchymal stem cells, or other adipocyte progenitors relevant to adipose tissue repair and research. The composition of the present invention can encourage adipogenesis of stem cells injected with it, as well as stem cells naturally present in the injection region. In some instances, the decellularized and delipidized adipose matrix of the present invention can also be used to coat implanted devices or materials to improve adipogenesis or biocompatibility around the device.

The present invention further provides a method of producing a composition comprising a decellularized and delipidized extracellular matrix derived from adipose or loose connective tissue, particularly from lipoaspirate obtained from liposuction. The inventive method comprises the following steps: obtaining an adipose tissue sample (e.g., lipoaspiratc) having an extracellular matrix component and non-extracellular matrix component; treating the adipose tissue sample with one or more decellularization agents, such as sodium dodecyl sulfate (SDS) or sodium deoxycholate or other detergents, to obtain decellularized adipose or loose connective tissue extracellular matrix comprising extracellular proteins (e.g., collagen I, II, III, and laminin) and polysaccharides (e.g., glycosaminoglycans). The invention further comprises treating the decellularized adipose or loose connective tissue extracellular matrix with one or more delipidizing agents, such as lipase and colipase, or other enzymes, to obtain decellularized and delipidized extracellular matrix. Finally, the method can include sterilizing the resulting decellularized and delipidized extracellular matrix. In some instances, the methods and use of detergents and lipase can also be utilized to decellularize and delipidize other tissue components that have lipids, such as skeletal muscle, heart, or liver.

In some instances, the method further comprises the step of freezing, lyophilizing and grinding up the decellularized and delipidized adipose or loose connective tissue extracellular matrix. In some instances, the method further comprises the step of enzymatically treating (e.g., with pepsin) the decellularized and delipidized adipose or loose connective tissue extracellular matrix, followed by a step of suspending and neutralizing the decellularized and delipidized adipose or loose connective tissue extracellular matrix in a solution to obtain a solubilized, decellularized and delipidized adipose or loose connective tissue extracellular matrix. In some instances, the method further comprises the step of re-lyophilizing the extracellular matrix solution and then rehydrating prior to injection or implantation.

In some instances, the decellularized adipose extracellular matrix is digested with pepsin at a low pH. In some instances, the solution is a phosphate buffered solution (PBS) or saline solution which can be injected through a 25 gauge needle or smaller into the adipose tissue. In some instances, the composition is formed into a gel in vivo at body temperature, and/or gelled, modified and modified to a desired shape ex vivo, and then implanted as a three-dimensional form. In some instances, said composition further comprises cells, drugs, proteins or other therapeutic agents that can be delivered within or attached to the composition before, during or after gelation.

The present invention further provides a method of providing to any individual an adipose or loose connective tissue matrix scaffold comprising parentally administering to or implanting into an individual in need thereof an effective amount of the composition or gel formation thereof, comprising the decellularized and delipidized adipose or loose connective tissue extracellular matrix. In some instances, the present invention also provides a method of encouraging adipogenesis of stem or progenitor cells injected or naturally present in the injection region using the decellularized and delipidized adipose or loose connective tissue extracellular matrix. In some instances, the present invention also provides a method of improving biocompatibility around implanted devices by coating the implanted devices with the decellularized and delipidized adipose or loose connective tissue extracellular matrix.

Furthermore, the present invention provides a method of culturing cells on an adsorbed matrix comprising the steps of providing a solution comprising decellularized and delipidized extracellular matrix derived from adipose or loose connective tissue into a tissue culture device; incubating the tissue culture device to adsorb at least some of the decellularized and delipidized extracellular matrix onto the device; removing the solution; and culturing exogenous cells on the adsorbed matrix. In some instances, the exogenous cells are adipocytes, lipoblasts, adipose-derived mesenchymal stem cells, adipose cell progenitors, and any other cell types relevant to adipose tissue repair or regeneration.

### BRIEF DESCRIPTION OF THE DRAWINGS

FIG. 1 illustrates production of decellularized and delipidized lipoaspirate. Human lipoaspirate was processed to remove both cellular and lipid content. Raw lipoaspirate (FIGS. 1A, 1D, 1G, 1J) was decellularized for 48 hours in SDS or sodium deoxycholate to produce a lipid filled, acellular matrix (FIGS. 1B, 1E, 1H, 1K). Removal of lipids using lipase produced a white ECM, free of cellular and lipid content (FIGS. 1C, 1F, 1I, 1L, not shown). H&E staining (FIGS. 1D, 1E, 1F) and Hoechst staining (not shown) confirmed the absence of nuclei after processing. Oil red O staining (FIGS. 1G, 1H, 1I) confirmed the removal of lipids. Scale bars=100 μm.

FIG. 2 illustrates quantification of remaining DNA. A DNEasy assay quantified the remaining nuclear content after decellularization and delipidization of the lipoaspirate. * p<0.0001.

FIG. 3 illustrates solubilization and gelation of adipose matrix. Decellularized and delipidized adipose matrix produced a dry, white powder (FIG. 3A) that was solubilized using pepsin and HCl (FIG. 3B). This solubilized adipose matrix was induced to self-assemble (FIG. 3C) when placed under physiologic conditions (37° C. and 5% CO₂).

FIG. 4 illustrates SDS-PAGE analysis of peptide content within the decellularized and delipidized adipose matrix. As compared to a collagen control (lane C), gel electrophoresis revealed collagen as well as multiple lower molecular weight peptides present within adipose matrix that had been decellularized using SDS (lane A) or sodium deoxycholate (lane B). Protein ladder (lane D) was run with peptide weights in kDa.

FIG. 5 illustrates an immunofluorescent staining of adipose matrix. Fluorescent antibody staining of both fresh human lipoaspirate (FIG. 5A) and adipose matrix decellularized with SDS (FIG. 5B) showed retention of collagens I, III, and IV. Laminin was also present in both cases, but there was some loss of content as a result of the decellularization. Scale bar=100 μm.

FIG. 6 illustrates a scanning electron microscopy of adipose matrix. SEM images of adipose matrix gels revealed a porous structure composed of intermeshed fibers with a diameter of approximately 100 nm. Scale bars=2 μm (FIG. 6A) and 500 nm (FIG. 6B).

FIG. 7 illustrates an in vitro culture of hASCs on 20 adipose matrix. Live/Dead analysis after 14 days in culture revealed negligible cell death of hASCs seeded on normal tissue culture plastic (FIG. 7A), calf skin collagen (FIG. 7B), or decellularized adipose matrix (FIG. 7C). Cells growing on the adipose matrix also exhibited a healthy fibroblast-like phenotype (FIG. 7D with F-actin and nuclei shown). PicoGreen analysis at various time points indicates that the adipose ECM promoted normal proliferation over 2 weeks in culture (FIG. 7E). Each group increased significantly between time points but no significant difference was found between groups at each time point. * p<0.0001 for Day 7 values for each group compared to Day 1 values. † p<0.0001 for Day 14 values for each group compared to Day 7 values. Scale bars=100 μm.

FIG. 8 illustrates an in vivo gelation of solubilized adipose matrix. Solubilized adipose matrix was injected subcutaneously into nude mice using a 25G needle (FIG. 8A). The solubilized ECM formed a solid bolus beneath the skin within 15 minutes (FIG. 8B). Gels held their shape when excised (FIG. 8C) and were analyzed with H&E (FIG. 8D). This staining showed an acellular matrix (m) in close contact with native fat (f). Scale bar=50 μm.

FIG. 9 illustrates upregulation of adipose related gene, apt expression in hASC when cultured on adsorbed adipose matrix coating. hASCs were cultured on either tissue culture plastic or adsorbed adipose matrix coating.

### DETAILED DESCRIPTION OF THE INVENTION

The present invention provides a composition comprising decellularized and delipidized extracellular matrix (ECM) derived from adipose or loose connective tissue, and methods of use thereof. The composition of the present invention can be used, for example, to support regeneration of adipocytes and to deliver therapeutic agents, including exogenous cells, into the tissue of a subject in need of therapeutic tissue engineering, filling soft tissue defects, or cosmetic and reconstructive procedures. The extracellular matrix of the invention can also be adapted for culturing cells ex vivo for further research or commercial purposes. The extracellular matrix of the present invention can be derived from the native or natural matrix of adipose, loose connective tissue or other tissues that contain adipocytes. The decellularized and delipidized extracellular matrix retains at least some native peptides and glycosaminoglycans which support regeneration of adipocytes. The decellularized and delipidized extracellular matrix retains at least some native peptides and glycosaminoglycans which support biological activity, such as regeneration of adipocytes or other bodily repair response.

Described herein are compositions comprising decellularized and delipidized adipose or loose connective tissue extracellular matrix which can be used for injection or surgical delivery into patients in need of treatment. The adipose or loose connective tissue extracellular matrix of the present invention can also be used to recruit the patients' cells into the injured tissue or as a cell or drug delivery vehicle, and can also be used to support injured tissue or change the mechanical properties of the tissue. Adipose or loose connective tissue extracellular matrix as described herein is derived from adipose or loose connective tissue, or other tissues containing adipocytes and lipids.

An injectable composition comprising the decellularized and delipidized adipose or loose connective tissue extracellular matrix as described herein provides the a scaffold specifically designed for adipose tissue that retains the tissue specific matrix properties important for native cell infiltration and transplanted cell survival and differentiation. The adipose or loose connective tissue extracellular matrix material can be used for autologous, allogenic or xenogenic treatments. By using decellularized and delipidized extracellular matrix, the composition mimics the extracellular environment present in adipose tissue such as by providing certain proteins such as collagens I, III and IV and glycosaminoglycans such as laminin. The invention encourages the migration of host progenitor cells that will regenerate new adipose tissue in vivo and aid integration with the existing tissue. The composition can also be modified to encourage biological processes such as angiogenesis by attaching growth factors to the binding receptors inherently present in the remaining extracellular matrix, which will enhance this new tissue formation.

The extracellular matrix composition is derived from adipose or loose connective tissue of an animal. An extracellular matrix composition herein can further comprise one or more additional components, for example without limitation: platelet-rich plasma (PRP) derived from whole blood, an exogenous cell, a polypeptide, a protein, a vector expressing a DNA of a bioactive molecule, and other therapeutic agents such as drugs, cellular growth factors, chemotaxis agents, nutrients, antibiotics or other bioactive molecules. Therefore, in certain preferred embodiments, the extracellular matrix composition can further comprise an exogenous population of cells such as adipocytes, lipoblasts, or precursors thereof, as described below.

In some instances, methods of delivery are described wherein the composition comprising the adipose extracellular matrix can be placed in contact with a defective, diseased or absent adipose or loose connective tissue, resulting in adipose and/or loose connective tissue repair or regeneration. In some instances, the composition comprising the adipose extracellular matrix herein can recruit endogenous cells within the recipient and can coordinate the function of the newly recruited or added cells, allowing for cell proliferation or migration within the composition.

The invention provides decellularized and delipidized adipose tissue extracellular matrix, as well as methods for the production and use thereof. In particular, the invention relates to a biocompatible composition comprising decellularized and delipidized extracellular matrix derived directly from lipoaspirate obtained from surgical liposuction of an adipose tissue. The composition can be used for treating defective, diseased, or damaged adipose tissue, loose connective tissues, or soft tissues or organs in a subject, including a human, by injecting or implanting the biocompatible composition comprising the decellularized and delipidized adipose extracellular matrix into the subject. Other embodiments of the invention concern decellularized and delipidized loose connective tissues containing adipocytes and lipids, extracellular matrix compositions made therefrom, methods of use and methods of production.

In some instances, the decellularized and delipidized adipose or loose connective tissue extracellular matrix is derived from native adipose or loose connective tissue selected from the group consisting of human, porcine, bovine, goat, mouse, rat, rabbit, or any other mammalian or animal fat or other adipose or loose connective tissue. In some embodiments, the biocompatible composition comprising the decellularized and delipidized adipose or loose connective tissue extracellular matrix is prepared into an injectable solution form, and can be used for adipose tissue or connective tissue repair by transplanting or delivering therapeutic agents or cells contained therein into the defective, diseased, or damaged tissues, or recruiting the patient's own cells into the extracellular matrix of the invention. In other instances, the biocompatible material comprising a decellularized and delipidized adipose or loose connective tissue extracellular matrix is, for example incorporated into another bodily implant, a patch, an emulsion, a viscous liquid, particles, microbeads, or nanobeads.

In some instances, the invention provides biocompatible materials for culturing adipocytes, lipoblasts or other adipose- or loose connective-tissue relevant cells, as well as other tissue-specific stem or progenitor cells, in research laboratories, or in a clinical setting prior to transplantation and for adipose or loose connective tissue repair or regeneration. Methods for manufacturing and coating a culture surface, such as tissue culture plates or wells, with decellularized and delipidized adipose or loose connective tissue extracellular matrix are also provided. The biocompatible materials of the invention are also suitable for implantation into a patient, whether human or animal.

The present invention further provides a native adipose or loose connective tissue extracellular matrix decellularization, delipidization, solubilization, and gelation method to create an in situ scaffold for cellular transplantation. An appropriate digestion and preparation protocol is provided that can create nanofibrous gels. The gel solution is capable of being injected or surgically implanted into the adipose or loose connective tissue, thus demonstrating its potential as an in situ gelling scaffold. The decellularized, delipidized, and solubilized extracellular matrix of the present invention can also be gelled ex vivo, modified and shaped if desired, and then implanted as a three-dimensional scaffold. Since a decellularized and delipidized adipose tissue extracellular matrix mimics the natural adipose or loose connective tissue environment, it improves cell survival and retention at the site, thus encouraging adipose or loose connective tissue regeneration.

In some instances, the methods can also be utilized to decellularize other tissues that have lipid components, such as skeletal muscle, heart, or liver. The resulting decellularized and delipidized extracellular matrix can be used as a material for adipose tissue engineering, filling soft tissue defects, and cosmetic and reconstructive surgery as non-limiting examples. The composition can be injected in particulate form or digested to create a solution that reassembles into a gel after injection. Implantation of the intact matrix as a gel formed, modified, and shaped ex vivo, is also possible. The material can be used alone to recruit cells and vasculature into the injection site, as a drug delivery vehicle, or in combination with other exogenous cells (e.g., human adipose derived stem cells) or plasma (e.g., the platelet-rich plasma (PRP)) to promote repair or regeneration. The decellularized and delipidized adipose extracellular matrix can also be used as a substrate to culture adipose derived stem cells, as well as other stem or progenitor cells, for research and commercial expansion.

In certain embodiments, the present invention provides a method of producing a composition comprising a decellularized and delipidized extracellular matrix derived from adipose or loose connective tissue, particularly, from lipoaspirate obtained from surgical liposuction. The method comprises the following steps: obtaining an adipose tissue sample having an extracellular matrix component and non-extracellular matrix adipocyte component; treating the adipose tissue sample with one or more decellularization detergent agents, such as sodium dodecyl sulfate (SDS) and sodium deoxycholate, to obtain decellularized adipose or loose connective tissue extracellular matrix, including extracellular proteins (e.g., collagen I, II, III, and laminin) and polysaccharides (e.g., glycosaminoglycans). Decellularization can be performed with a perfusion of one or more decellularization agents, such as detergents, sodium dodecyl sulfate (SDS), sodium deoxycholate, and TRITON X-100 (C₁₄H₂₂O(C₂H₄O)ₙ), and peracetic acid, alone or in combination, for example. Other decellularization agents include, but are not limited to, TRITON X-200, 3-[(3-cholamidopropyl)dimethylammonio]-1-propanesulfonate (CHAPS), 3-[(3-cholamidopropyl)-dimethylammonio]-2-hydroxy-1-propanesulfonate (CHAPSO), Sulfobetaine-10 (SB-10), Sulfobetaine-16 (SB-16), Tri(n-butyl)phosphate, Ethylenediaminetetraacetic acid (EDTA), and Ethylene glycol tetraacetic acid (EGTA). An alternation of hypertonic and hypotonic solutions could also be used, alone or in combination, with the above agents for decellularization. The compositions comprise an adipose tissue extracellular matrix that is decellularized in that the majority of living cells in the adipose or loose connective tissue are removed. In some instances, a substantially decellularized matrix comprises less than 25%, 20%, 15%, 10%, 9%, 8%, 7%, 6%, 5%, 4%, 3%, 2%, or 1% of original adipocyte cellular DNA from the donor tissue. The amount of decellularization can be determined indirectly through an analysis of DNA content remaining in the decellularized adipose extracellular matrix, as described herein.

The method involves further treating the decellularized adipose or loose connective tissue extracellular matrix with one or more delipidizing enzymatic agents, such as lipase or colipase, to obtain decellularized and delipidized extracellular matrix. Alternative delipidization agents that can be used alone or in combination with the above enzymes include, but are not limited to, endonucleases, exonucleases, DNase, RNase, or organic/polar solvents (e.g., acetone, hexane, cyclohexane, dichloromethane, isopropanol, ethanol). The compositions comprise a decellularized matrix that is also substantially delipidized in that the majority of the lipids in the adipose or loose connective tissue are removed. In some instances, a delipidized matrix comprises less than 25%, 20%, 15%, 10%, 9%, 8%, 7%, 6%, 5%, 4%, 3%, 2%, or 1% of native lipid from the donor tissue. The amount of delipidization can be determined indirectly through an oil imagine staining or a visual inspection of the whitening of the tissue, as described herein.

The adipose or loose connective tissue extracellular matrix can then be freeze-dried or lyophilized, and milled. The ground extracellular matrix can be solubilized with an aqueous solution such as water or saline, for example. In some embodiments, the extracellular matrix can be solubilized at a low pH, between about pH 1-6, or pH 1-4 such as through addition of HCl. In some embodiments, the matrix is digested with pepsin or alternative matrix peptide or glycosaminoglycan digesting enzymes, such as papain, matrix metalloproteinases, collagenases, and trypsin. In some instances, the method further comprises the step of re-lyophilizing the extracellular matrix solution, and then rehydrating in an aqueous solution prior to injection or implantation.

To produce a gel form of the adipose or loose connective tissue extracellular matrix for in vivo therapy, the solution comprising the adipose or loose connective tissue extracellular matrix can then be neutralized and brought up to the desired temperature, concentration and viscosity using PBS/saline. Depending upon the concentration of proteins and glycosaminoglycans in a particular sample, and the amounts of matrix digestive enzymes used, the resulting extracellular matrix composition can be routinely solubilized for a desired gelling formation at temperatures greater than 20° C., 25° C., 30° C., or 35° C., and over a period of time, including from less than 30, 20, 10, 5, or 1 minutes. In some embodiments, the extracellular matrix comprises digested proteins and/or glycosaminoglycans with an average molecular weight of less than 300 kDa, 200 kDa, 100 kDa, 50 kDa, or less than 20 kDa.

In certain embodiments, the extracellular matrix concentration can be 1-100 mg/mL, 2-8 mg/mL, 10 mg/mL, 20 mg/mL, 30 mg/mL, 40 mg/mL, 50 mg/mL, 60 mg/mL, 70 mg/mL, 80 mg/mL, 90 mg/mL, and 100 mg/mL as desired to effect viscosity. The solution comprising the adipose or loose connective tissue extracellular matrix can then be injected through a needle, such as 25 gauge or smaller, into the desired site of a subject in need.

Cells, plasma, drugs, proteins, or other biologically active agents can also be delivered inside the adipose or loose connective tissue extracellular matrix gel. Decellularized and delipidized extracellular matrices are prepared such that natural or enhanced bioactivity for the adipose or loose connective tissue matrix is established. Exemplary bioactivity of the compositions herein include without limitation: cell adhesion, cell migration, cell differentiation, cell maturation, cell organization, cell proliferation, cell death (apoptosis), stimulation of angiogenesis, proteolytic activity, enzymatic activity, cell motility, protein and cell modulation, activation of transcriptional events, provision for translation events, or inhibition of some bioactivities, for example inhibition of coagulation, stem cell attraction, chemotaxis, inflammation, immune response, bacterial growth, and MMP or other enzyme activity.

As described herein, a composition can comprise a decellularized and delipidized adipose or loose connective tissue extracellular matrix and exogenous synthetic or naturally occurring polymer and/or protein components useful for adipose tissue engineering or soft tissue repair. Exemplary polymers and/or protein components herein include, but are not limited to: polyethylene terephthalate fiber (DACRON), polytetrafluoroethylene (PTFE), glutaraldehyde-cross linked pericardium, polylactate (PLA), polyglycol (PGA), hyaluronic acid (HA), polyethylene glycol (PEG), polyethelene, nitinol, collagen from animal and non-animal sources (such as plants or synthetic collagens), fibrin, fibrinogen, thrombin, alginate, chitosan, silk, proteins extracted from cultured adipocytes or adipose derived stem cells (ASCs), platelet rich plasma (PRP), and carboxymethyl cellulose. In some instances, a polymer added to the composition is biocompatible, biodegradable or bioabsorbable. Exemplary biodegradable or bioabsorbable polymers include, but are not limited to: polylactides, poly-glycolides, polycarprolactone, polydioxane and their random and block copolymers. A biodegradable or bioabsorbable polymer can contain a monomer selected from the group consisting of a glycolide, lactide, dioxanone, caprolactone, trimethylene carbonate, ethylene glycol and lysine.

The polymer material can be a random copolymer, block copolymer or blend of monomers, homopolymers, copolymers, and/or heteropolymers that contain these monomers. The biodegradable and/or bioabsorbable polymers can contain bioabsorbable and biodegradable linear aliphatic polyesters such as polyglycolide (PGA) and its random copolymer poly(glycolide-co-lactide-) (PGA-co-PLA). Other examples of suitable biocompatible polymers are polyhydroxyalkyl methacrylates including ethylmethacrylate, and hydrogels such as polyvinylpyrrolidone and polyacrylamides. Other suitable bioabsorbable materials are biopolymers which include collagen, gelatin, alginic acid, chitin, chitosan, fibrin, hyaluronic acid, dextran, polyamino acids, polylysine and copolymers of these materials. Any combination, copolymer, polymer or blend thereof of the above examples is contemplated for use according to the present invention.

In certain embodiments, the viscosity of the composition increases when warmed above room temperature including physiological temperatures approaching about 37° C. According to one non-limiting embodiment, the extracellular matrix-derived composition is an injectable solution at room temperature and other temperatures below 35° C. In another non-limiting embodiment the gel can be injected at body temperature, but gels more rapidly at increasing temperatures. In certain embodiments, a gel can form after approximately 1-30 or 15-20 minutes at physiological temperature of 37° C. Principles for preparing an extracellular matrix-derived gel are provided along with preferred specific protocols for preparing gels, which are applicable and adaptable by those of skill in the art according to the needs of a particular situation and for numerous tissues including without limitation adipose or loose connective tissues.

The decellularized and delipidized compositions which may include exogenous cells or other therapeutic agents may be implanted into a patient, human or animal, by a number of methods. In some instances, the compositions are injected as a liquid into a desired site in the patient which then spontaneously gels in situ at approximately 37° C.

The compositions herein provide a gel or solution form of adipose or loose connective tissue extracellular matrix, and the use of these forms of extracellular matrix for adipose or loose connective tissue engineering, filling of soft tissue defects, and cosmetic and reconstructive surgery. In one embodiment, the adipose or loose connective tissue is first decellularized, leaving only the extracellular matrix, and then delipidized. In alternative embodiments, the tissue can first be delipidized, then decellularized, or the tissue can be simultaneously delipidized and decellularized. The decellularized and delipidized matrix can then be freeze-dried or lyophilized, then milled, ground or pulverized into a fine powder, and solubilized with pepsin or other enzymes, such as, but not limited to, matrix metalloproteases, collagenases, and trypsin.

For gel therapy, the solution can be neutralized and brought up to the appropriate concentration using PBS/saline. In one embodiment, the solution can then be injected through a needle or delivered into the desired site using any delivery methods known in the art. The needle size can be without limitation 22G, 23G, 24G, 25G, 26G, 27G, 28G, 29G, 300, 31 G, 32 G, or smaller. In one embodiment, the needle size through which the solution is injected is 25G. Dosage amounts and frequency can routinely be determined based on the varying condition of the injured tissue and patient profile. At body temperature, the solution can then form into a gel. In yet another embodiment, the solution and/or gel can be crosslinked with glutaraldehye, EDC, transglutaminase, formaldehyde, bis-NHS molecules, or other crosslinkers to increase material stiffness and modulate degradation of the material.

In yet another embodiment, the extracellular matrix can be combined with other therapeutic agents, such as cells, peptides, proteins, DNA, drugs, nutrients, antibiotics, survival promoting additives, proteoglycans, and/or glycosaminolycans. In yet another embodiment, the extracellular matrix can be combined and/or crosslinked with a natural or synthetic polymer.

In yet another embodiment, extracellular matrix solution or gel can be injected into the affected site or area alone or in combination with above-described components for endogenous cell ingrowth, angiogenesis, and regeneration. In yet another embodiment, the composition can also be used alone or in combination with above-described components as a matrix to change mechanical properties of the adipose and/or loose connective tissue. In yet another embodiment, the composition can be delivered with cells alone or in combination with the above-described components for regenerating adipose or loose connective tissue. In yet another embodiment, the composition can be used alone or in combination with above-described components for filling soft tissue and/or cosmetic or reconstructive surgery. In yet another embodiment, the composition can be used to coat implanted devices or materials to improve adipogenesis or biocompatibility around the devices.

In one embodiment for making a soluble reagent, the solubilized matrix is brought up in a low pH solution including but not limited to 0.5 M, 0.1M, or 0.01M acetic acid or 0.1M HCl to the desired concentration and then placed into tissue culture plates/wells, coverslips, scaffolding or other surfaces for tissue culture. After placing in an incubator at 37° C. for 1 hour, or overnight at room temperature, or overnight at 2-4° C., the excess solution is removed. After the surfaces are rinsed with PBS, cells can be cultured on the adsorbed matrix. The solution can be combined in advance with peptides, proteins, DNA, drugs, nutrients, survival promoting additives, platelet-rich plasma (PRP), proteoglycans, and/or glycosaminoglycans.

The present invention provides enhanced cell attachment and survival in both the therapeutic composition and adsorbed cell culturing composition forms of the adipose or loose connective tissue extracellular matrix in vitro. The soluble cell culturing reagent form of the adipose or loose connective extracellular matrix induces faster spreading, faster maturation, and/or improved survival for adipocytes or lipoblasts compared to standard plate coatings. The extracellular matrix can also cause cellular differentiation of stem or progenitor cells.

In an embodiment herein, a biomimetic matrix derived from native adipose or loose connective tissue is disclosed. In some instances, a matrix resembles the in vivo adipose or loose connective tissue environment in that it contains many or all of the native chemical cues found in natural adipose or loose connective extracellular matrix. In some instances, through crosslinking or addition or other materials, the mechanical properties of healthy adult or embryonic adipose or loose connective tissue can also be mimicked. As described herein, adipose or loose connective tissue extracellular matrix can be isolated and processed into a gel using a simple and economical process, which is amenable to scale-up for clinical translation.

In some instances, a composition as provided herein can comprise a matrix and exogenously added or recruited cells. The cells can be any variety of cells. In some instances, the cells are a variety of adipocyte, lipoblast, or related cells including, but not limited to: stem cells, progenitors, adipocytes, lipoblasts, and fibroblasts derived from autologous or allogeneic sources.

The invention thus provides a use of a gel made from native decellularized and delipidized adipose or loose connective extracellular matrix to support isolated neonatal adipocytes or lipoblasts or stem cell progenitor derived adipocytes or lipoblasts in vitro and act as an in situ gelling scaffold, providing a natural matrix to improve cell retention and survival in the adipose or loose connective tissue. A scaffold created from adipose or loose connective extracellular matrix is well-suited for cell transplantation in the adipose or loose connective tissue, since it more closely approximates the in vivo environment compared to currently available materials.

A composition herein comprising adipose or loose connective tissue extracellular matrix and exogenously added cells can be prepared by culturing the cells in the extracellular matrix. In addition, where proteins such as growth factors are added into the extracellular matrix, the proteins may be added into the composition, or the protein molecules may be covalently or non-covalently linked to a molecule in the matrix. The covalent linking of protein to matrix molecules can be accomplished by standard covalent protein linking procedures known in the art. The protein may be covalently or linked to one or more matrix molecules.

In one embodiment, when delivering a composition that comprises the decellularized and delipidized adipose or loose connective tissue extracellular matrix and exogenous cells, the cells can be from various cell sources including autogenic, allogenic, or xenogenic, sources. Accordingly, embryonic stem cells, fetal or adult derived stem cells, induced pluripotent stem cells, adipocyte or lipoblast progenitors, fetal and neonatal adipocytes or lipoblasts, adipose-fibroblasts, mesenchymal cells, parenchymal cells, epithelial cells, endothelial cells, mesothelial cells, fibroblasts, hematopoietic stem cells, bone marrow-derived progenitor cells, skeletal cells, smooth muscle cells, macrophages, cardiocytes, myofibroblasts, and autotransplanted expanded adipocytes can be delivered by a composition herein. In some instances, cells herein can be cultured ex vivo and in the culture dish environment differentiate directly or indirectly to adipose or loose connective tissue cells. The cultured cells are then transplanted into the mammal, either alone or in contact with the scaffold and other components.

Adult stem cells are yet another species of cell that can be part of a composition herein. Adult stem cells are thought to work by generating other stem cells in a new site, or they differentiate directly or indirectly to an adipocyte in vivo. They may also differentiate into other lineages after introduction to organs. The adult mammal provides sources for adult stem cells, circulating endothelial precursor cells, bone marrow-derived cells, adipose tissue, or cells from a specific organ. It is known that mononuclear cells isolated from bone marrow aspirate differentiate into endothelial cells in vitro and are detected in newly formed blood vessels after intramuscular injection. Thus, use of cells from bone marrow aspirate can yield endothelial cells in vivo as a component of the composition. Other cells which can be employed with the invention are the mesenchymal stem cells administered, in some embodiments with activating cytokines. Subpopulations of mesenchymal cells have been shown to differentiate toward myogenic or adipogenic cell lines when exposed to cytokines in vitro.

Human embryonic stem cell derived or adult induced stem cells which can differentiate into adipocytes or lipoblasts can be grown on a composition herein comprising an adipose extracellular matrix. In some instances, hESC-derived adipocytes grown in the presence of a composition herein provide a more in vivo-like morphology. In some instances, hESC-derived adipocytes grown in the presence of a composition herein provide increased markers of maturation.

The invention is also directed to a drug delivery system comprising decellularized and delipidized adipose or loose connective tissue extracellular matrix for delivering cells, plasma, drugs, molecules, or proteins into a subject for treating defective, diseased, or damaged tissues or organs, or for filling soft tissue and cosmetic and reconstructive surgery. The inventive biocompatible material can be used to transplant cells, or injected alone to recruit native cells or other cytokines endogenous therapeutic agents, or act as an exogenous therapeutic agent delivery vehicle.

The composition of the invention can further comprise proteins, or other biological material such as, but not limited to, erythropoietin (EPO), stem cell factor (SCF), vascular endothelial growth factor (VEGF), transforming growth factor (TGF), fibroblast growth factor (FGF), epidermal growth factor (EGF), cartilage growth factor (CGF), nerve growth factor (NGF), keratinocyte growth factor (KGF), skeletal growth factor (SGF), osteoblast-derived growth factor (BDGF), hepatocyte growth factor (HGF), insulin-like growth factor (IGF), cytokine growth factor (CGF), stem cell factor (SCF), platelet-derived growth factor (PDGF), endothelial cell growth supplement (EGGS), colony stimulating factor (CSF), growth differentiation factor (GDF), integrin modulating factor (IMF), calmodulin (CaM), thymidine kinase (TK), tumor necrosis factor (TNF), growth hormone (GH), bone morphogenic proteins (BMP), matrix metalloproteinase (MMP), tissue inhibitor matrix metalloproteinase (TIMP), interferon, interleukins, cytokines, integrin, collagen, elastin, fibrillins, fibronectin, laminin, glycosaminoglycans, hemonectin, thrombospondin, heparin sulfate, dermantan, chondroitin sulfate (CS), hyaluronic acid (HA), vitronectin, proteoglycans, transferrin, cytotactin, tenascin, lymphokines, and platelet-rich plasma (PRP).

Tissue culture plates can be coated with either a soluble ligand or gel form of the extracellular matrix of the invention, or an adsorbed form of the extracellular matrix of the invention, to culture adipocytes, lipoblasts, or other cell types relevant to adipose or loose connective tissue repair or regeneration. This can be used as a research reagent for growing these cells or as a clinical reagent for culturing the cells prior to implantation. The extracellular matrix reagent can be combined with other tissue matrices and cells.

For gel reagent compositions, the solution is then neutralized and brought up to the appropriate concentration using PBS/saline or other buffer, and then be placed into tissue culture plates and/or wells. Once placed in an incubator at 37° C., the solution forms a gel that can be used for any two- or three-dimensional culture substrate for cell culture. In one embodiment, the gel composition can be crosslinked with glutaraldehye, formaldehyde, bis-NHS molecules, or other crosslinkers, or be combined with cells, peptides, proteins, DNA, drugs, nutrients, survival promoting additives, proteoglycans, and/or glycosaminolycans, or combined and/or crosslinked with a synthetic polymer for further use.

The invention further provides an exemplary method of culturing cells adsorbed on a decellularized and delipidized adipose or loose connective tissue extracellular matrix comprising the steps of: (a) providing a solution comprising the biocompatible material of decellularized and delipidized extracellular matrix derived from adipose or loose connective tissue in low pH solution, including but not limited to, 0.5 M, or 0.01M acetic acid or 0.1M HCl to a desired concentration, (b) placing said solution into a tissue culture device, such as plates or wells, (c) incubating said tissue culture plates or wells above room temperature such as at 37° C., for between 1 hour and twelve hours incubation at 2-4° C. or up to room temperature to 40° C. to adsorb at least some of the decellularized and delipidized extracellular matrix onto the plates or wells, (d) removing excess solution, (e) rinsing said tissue culture plates or wells with PBS, and (f) culturing cells on the adsorbed matrix. Cells that can be cultured on the adsorbed matrix comprising the adipose or loose connective tissue extracellular matrix of the invention include adipocytes, lipoblasts, or other cell types relevant to adipose or loose connective tissue repair or regeneration, including stem cells and adipose or loose connective tissue progenitors.

In one instance, a composition can include a bioadhesive, for example, for wound repair. In some instances, a composition herein can be configured as a cell adherent. For example, the composition herein can be coated on or mixed with a medical device or a biologic that does or does not comprise cells. Methods herein can comprise delivering the composition as a wound repair device.

In some instances, the composition is injectable. An injectable composition can be, without limitation, a powder, liquid, particles, fragments, gel, or emulsion. The injectable composition can be injected into a desired site comprising defective, diseased, or damaged adipose or loose connective tissue. The compositions herein can recruit, for example without limitation, endothelial, smooth muscle, adipocyte or lipoblast progenitors, fibroblasts, and stem cells.

Methods herein include delivery of a composition comprising an extracellular matrix by methods well known in the art. The composition can also be delivered in a solid formulation, such as a graft or patch or associated with a cellular scaffold. Dosages and frequency will vary depending upon the needs of the patient and judgment of the physician.

In some instances, a decellularized and delipidized extracellular matrix derived from adipose or loose connective tissue composition herein is a coating. A coating can be used for tissue culture applications, both research and clinical. The coating can be used to coat, for example without limitation, synthetic or other biologic scaffolds/materials, or implants. In some instances, a coating is texturized or patterned. In some instances, a method of making a coating includes adsorption or chemical linking. A thin gel or adsorbed coating can be formed using an ECM solution form of the composition.

The extracellular matrix consists of a complex tissue-specific network of proteins and polysaccharides, which help regulate cell growth, survival and differentiation. Despite the complex nature of native extracellular matrix, in vitro cell studies traditionally assess cell behavior on single extracellular matrix component coatings, thus posing limitations on translating findings from in vitro cell studies to the in vivo setting. Overcoming this limitation is important for cell-mediated therapies, which rely on cultured and expanded cells retaining native cell behavior over time.

Typically, purified matrix proteins from various animal sources are adsorbed to cell culture substrates to provide a protein substrate for cell attachment and to modify cellular behavior. However, these approaches do not provide an accurate representation of the complex microenvironment. More complex coatings have been used, such as a combination of single proteins, and while these combinatorial signals have shown to affect cell behavior, it is not as complete as in vivo. For a more natural matrix, cell-derived matrices can be used. While many components of extracellular matrix are similar, each tissue or organ has a unique composition, and a tissue specific naturally derived source may prove to be a better mimic of the cell microenvironment.

In one aspect, a composition herein comprises extracellular matrix that is derived from adipose or loose connective tissue. The composition can be developed for substrate coating for a variety of applications. In some instances, the extracellular matrix of the composition retains a complex mixture of adipose-specific extracellular matrix components after solubilization. In some instances, the compositions can form coatings to more appropriately emulate the native adipose or loose connective extracellular matrix in vitro.

The invention is further illustrated by the following examples, which are not to be construed in any way as imposing limitations upon the scope thereof. It is apparent for skilled artisans that various modifications and changes are possible and are contemplated within the scope of the current invention.

### Examples

### Materials and Methods

### Collection of Source Material and Cell Isolation

Fresh human lipoaspirate was collected from female patients, ranging from 39-58 years of age with an average age of 43, undergoing elective liposuction surgery under local anesthesia at the La Jolla Plastic & Reconstructive Surgery Clinic (La Jolla, Calif.) with the approval of the UCSD Institutional Review Board. Adipose-derived mesenchymal stem cells (hASCs) were first isolated from the tissue according to established protocols [34, 35]. Briefly, the tissue was digested in 0.075% collagenase I (Worthington Biochemical Corp., Lakewood, N.J.) for 20 minutes and the resulting suspension was centrifuged at 5000×g. The hASC-rich pellet was resuspended in 160 mM ammonium chloride to lyse blood cells and again centrifuged at 5000×g. The remaining cells were filtered and resuspended in Growth Medium (Dulbecco's modified essential medium/Ham's F12 (DMEM/F12, Mediatech, Manassas, Va.), 10% fetal bovine serum (FBS, Gemini Bio-Products, Sacramento, Calif.), and 100 I.U. penicillin/100 μg/mL streptomycin) and cultured overnight on standard tissue culture plastic at 37° C. and 5% CO₂. After 24 hours, non-adherent cells were removed with two rinses in 1× phosphate-buffered saline (PBS) and the remaining cells were serially passaged as hASCs. Growth Medium was changed every 3-4 days. When cells reached 80% confluence they were washed with 1×PBS and released from the tissue culture surface using 0.25% Trypsin/2.21 mM EDTA (Mediatech, Manassas, Va.). The cells were resuspended, counted, and plated in new flasks with fresh Growth Medium. The lipoaspirate not used for cell isolation was immediately stored at −80° C. and kept frozen until further processing.

### Decellularization and Delipidization of Human Lipoaspirate

Frozen lipoaspirate was slowly warmed to room to temperature and washed in 1×PBS for 2 hours under constant stirring. The PBS was then strained and the washed adipose tissue was placed in either 1% sodium dodecyl sulfate (SDS) in distilled water or 2.5 mM sodium deoxycholate in 1×PBS. Both of these detergents have been previously shown to be effective decellularization agents [36-38]. The tissue was stirred in detergent for 48 hours and subsequently thoroughly rinsed with DI water. Each group of decellularized tissue was then placed in 2.5 mM sodium deoxycholate in 1×PBS supplemented with 500 units of porcine lipase and 500 units of porcine colipase (both from Sigma-Aldrich, St. Louis, Mo.) to remove remaining lipids. This enzymatic digestion was continued until the tissue became visibly white, approximately 24-48 hours depending on the patient, or for a maximum of 72 hours if there was no change in color. Finally, the tissue was rinsed with DI water for 2 hours to remove excess detergents and frozen at −80° C. overnight. Prior to freezing, representative samples were embedded in Tissue Tek OCT compound for histological analysis. Following the decellularization and delipidization procedure, the frozen adipose-derived extracellular matrix was then lyophilized and milled using a Wiley Mini Mill.

### Evaluation of Decellularization and Delipidization

To examine the extent of decellularization of the adipose tissue, both fresh and decellularized samples that had been embedded in OCT were sectioned into 20 μm slices and stained with hematoxylin and eosin (H&E) for histological analysis. Decellularization was confirmed by staining slides with Hoechst 33342, a fluorescent nuclear stain. The tissue sections were fixed in acetone, rinsed, and stained in Hoechst dye at 0.1 μg/mL for 10 minutes. The sections were then rinsed, mounted with Fluoromount (Sigma-Aldrich, St. Louis, Mo.), and imaged with a Carl Zeiss Observer DI. Decellularization was further quantified using a commercially available DNEasy kit (Qiagen, Valencia, Calif.). Samples of lyophilized adipose matrix were weighed and DNA was extracted according to manufacturer's specifications. DNA content (μg/mg dry weight ECM) was estimated from absorbance readings at 260 nm using a BioTek Synergy H4 microplate reader (Winooski, VT) and normalized to initial dry weight of the sample. As a control, lyophilized calf skin collagen (Sigma-Aldrich, St. Louis, Mo.) was included in the assay.

Lipid removal from the tissue was assessed by staining with Oil Red O dye (Sigma-Aldrich, St. Louis, Mo.), as previously described [39]. Sections of fresh tissue and decellularized tissue, both before and after lipase treatment, were fixed with 3.2% paraformaldehyde for 1 hour and rinsed in DI water and then 60% isopropanol. Oil Red O stain was prepared at 5 mg/mL in 100% isopropanol and diluted 3:2 with DI water to make a working solution prior to use. Fixed tissue sections were stained in Oil Red O working solution for 15 minutes, rinsed in 60% isopropanol and then DI water, and mounted with 10% glycerol in 1×PBS. Images of the staining were taken using a Carl Zeiss Imager.

### Solubilization and Gelation of Decellularized Adipose Matrix

Dry, milled adipose matrix was further processed using 0.1M HCl and 3200 I.U. porcine pepsin (Sigma-Aldrich, St. Louis, Mo.), following a modified version of previously established protocols for different tissues [36, 40]. The pepsin was first solubilized in 0.1 M HCl and added to the adipose matrix at a ratio of 1 mg pepsin for every 10 mg lyophilized ECM. The adipose matrix was digested for 48 hours at room temperature under constant stirring. Subsequently, the pH was raised to 7.4 using 1 M NaOH and the matrix was diluted to 15 mg/mL using 10×PBS so that the final solution contained 1×PBS. This digest was kept on ice until used for characterization assays or gelation studies in vitro or in vivo. To induce gelation in vitro, the solubilized, neutralized adipose matrix was warmed to 37° C. in a humidified incubator with 5% CO₂. In vitro gels were characterized using an AR-G2 rheometer (TA Instruments, New Castle, Del.) with a 20 mm diameter parallel plate configuration. Gels produced from tissue decellularized with SDS and with sodium deoxycholate were tested at 37° C. under a constant 2.5% strain at an oscillating angular frequency of 1 rad/s.

### Characterization of Adipose Matrix

Peptide content of the solubilized adipose matrix was assessed using SDS-PAGE. Samples were run on a NUPAGE® Novex Bis-Tris gel (Invitrogen, Eugene, Oreg.) at 12% w/v in NUPAGE MOPS SDS running buffer (Ynvitrogen) and compared to rat tail collagen type I (2 mg/mL; BD Biosciences, San Jose, Calif.). Samples were prepared under reducing conditions with NuPAGE LDS Sample Buffer (Invitrogen) and run in an XCell Surelock MiniCell (Invitrogen) at a constant 200 V. Peptide bands were visualized using Imperial Protein Stain (Pierce, Rockford, Ill.). NOVEX® Plus2 Pre-stained Standard (Invitrogen) was used as a protein ladder. Sulfated glycosaminoglycan content of the adipose matrix was quantified using a colorimetric Blyscan assay (Biocolor, Carrickfergus, United Kingdom) according to manufacturer's instructions. Samples from different batches of adipose matrix were tested in triplicate and absorbance was recorded at 656 nm using a BioTek Synergy H4 microplate reader (Winooski, Vt.).

Immunofluorescent staining was used to identify specific proteins within the adipose matrix. Sections of both fresh lipoaspirate and adipose matrix were fixed with acetone and blocked with staining buffer (0.3% Triton X-100 and 2% goat serum in PBS). Samples were then stained with primary antibodies against collagen I, collagen III, collagen IV, and laminin (1:100 dilution, Abeam, San Francisco, Calif.). AlexaFluor 488 (1:200 dilution, Invitrogen) served as a secondary antibody. Both primary and secondary antibodies were individually omitted on control slides to confirm positive staining. Slides were mounted with Fluoromount (Sigma-Aldrich) and images were taken with a Carl Zeiss Observer DI.

Scanning electron microscopy was used to visualize the microstructure of adipose matrix gels. Gels were formed by warming solubilized adipose matrix to 37° C. in a humidified incubator with 5% CO₂ overnight. Gels were immersed in 2.5% gluteraldehyde for 2 hours and then dehydrated in a series of 15-minute ethanol rinses (30-100%) according to previously published protocols [21, 25, 40]. The gels were then critical point dried using CO, and coated with chromium using an Emitech K575X sputter coater. Scanning electron microscope images were taken using a Philips XL30 field emission SEM.

### In Vitro Cytocompatibility Assessment of Adipose Matrix

Solubilized adipose matrix was diluted to 5 mg/mL using 0.1M acetic acid and added to the bottom of wells of a 48-well tissue culture plate. The plate was kept at 4° C. overnight to adsorb the matrix to the tissue culture plastic. Control wells were either left as normal tissue culture plastic or coated with 1 mg/mL calf skin collagen solubilized in 0.1 M acetic acid. The leftover coatings were then aspirated and the wells were washed twice with 1×PBS. Passage 1 hASCs were seeded at 5×10⁴ cells/cm² in Growth Medium. Media was changed every 2-3 days. After 1, 7 and 14 days, cells were stained with a fluorescent Live/Dead Viability/Cytotoxicity Kit (Invitrogen, Carlsbad, Calif.). A solution of 4 μM calcein and 2 μM ethidium homodimer (EthD-1) was prepared in PBS. The solution was added to the cells and allowed to incubate for 30-45 minutes at room temperature. The cells were subsequently rinsed twice with PBS and then observed under a fluorescent microscope to examine the viability of the cells.

Total DNA content was assessed at each time point as well using the Quant-IT PicoGreen dsDNA Assay Kit (Invitrogen, Carlsbad, Calif.) to determine cellular proliferation. Briefly, the cells were rinsed twice in PBS and frozen at −20° C. for up to 1 week to aid cell lysis. Cellular DNA was then resuspended in 1×TE Buffer and incubated with a fluorescent PicoGreen Reagent for 30 minutes. Fluorescence was measured using a BioTek microplate reader with an excitation wavelength of 480 nm and emission wavelength of 520 nm. dsDNA was quantified by relating the sample absorbance to the absorbance measured for standards of known DNA concentration.

hASC morphology was visualized at each timepoint. Cells were washed with 1×PBS and fixed in 4% paraformaldehyde for 15 minutes. The cells were washed again and staining buffer (0.3% Triton X-100 and 1% bovine serum albumin in PBS) was added for 30 minutes to block non-specific binding. Cells were then incubated in AlexaFluor 488 Phalloidin (Invitrogen; 1:40 dilution in staining buffer) for 20 minutes to label F-actin and Hoechst 33342 (1 μg/mL in water) for 10 minutes to label nuclei. Images of the cells were taken using a Zeiss Observer DI.

### Subcutaneous Injection and Gelation of Solubilized Adipose Matrix

All animal procedures were performed in accordance with the guidelines established by the Committee on Animal Research at the University of California, San Diego and the American Association for Accreditation of Laboratory Animal Care. Male athymic mice (nu/nu) received an overdose of sodium pentobarbital and kept on heating pads. Solubilized and neutralized adipose matrix was drawn into a syringe using a 25 G needle. Six injections (100 μL each) were made subcutaneously into the dorsal region of the mouse. After 15 minutes, the injected material was excised and fresh frozen in TissueTek OCT compound. This tissue was then sectioned into 20 μm slices, stained with H&E for histological analysis, and examined using a Carl Zeiss Imager A1.

### Statistical Analysis

All data is presented as the mean±standard deviation. Both the Blyscan and DNEasy assays were performed in triplicate and the results averaged. Significance was assessed using one-way analysis of variance (ANOVA) and pose hoc analysis using either Dunnett's test or Tukey's test.

### Results

Isolation of Adipose ECM from Human Lipoaspirate

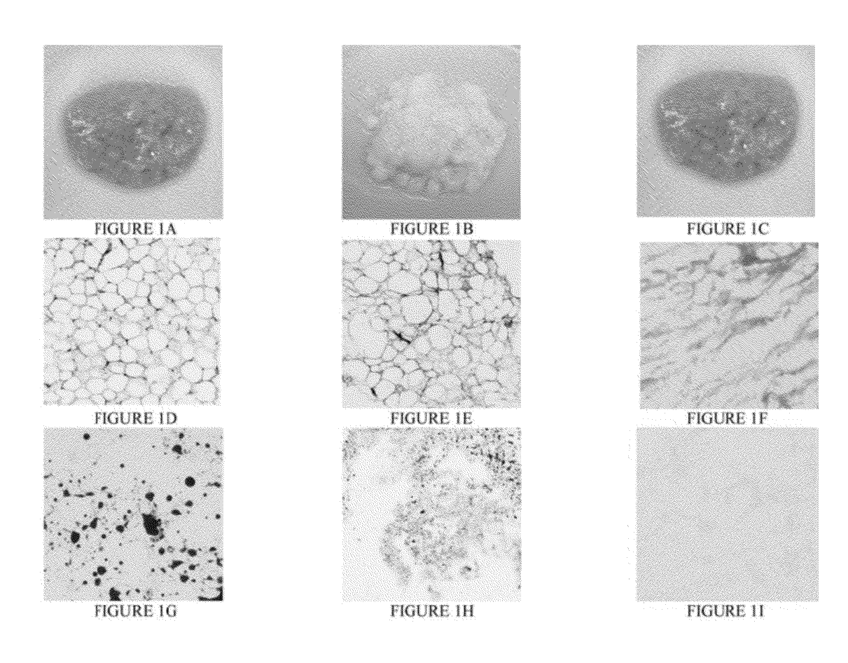

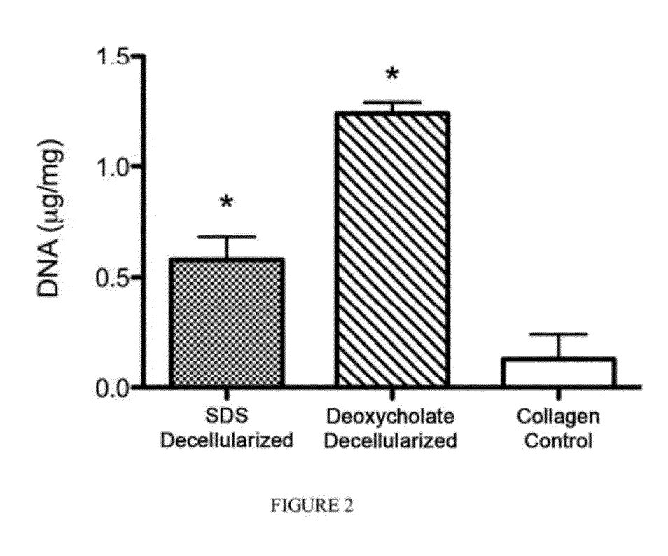

Fresh-frozen lipoaspirate was decellularized and delipidized within 4 days using a combined detergent and enzymatic digestion protocol. These methods were successfully repeated on samples from multiple patients, with the only variability arising in lipase digestion time (24-48 hours) due to initial lipid content. Average yield was 625±96 mg of dry adipose ECM per 100 cc of lipoaspirate (n=8). The use of either SDS or sodium deoxycholate were compared for decellularization, in combination with lipase and colipase for delipidization. Decellularization was confirmed by absence of nuclei with H&E and Hoechst 33342 in both the SDS and sodium deoxycholate groups (FIG. 1). While histological analysis demonstrated similar removal of cellular contents, a DNEasy kit revealed that SDS was more efficient in decellularizing the adipose ECM (FIG. 2), with significantly less DNA per mg of lyophilized ECM compared to the sodium deoxycholate group, and more closely approaching the collagen control.

After decellularization, removal of lipids was achieved through the addition of lipase and colipase for 24-48 hours, producing a white ECM compared to the characteristic yellow tint of adipose tissue. As seen in FIG. 1, Oil Red O staining of tissue sections revealed substantial levels of oils within fresh tissue, however treatment with lipase effectively removed lipids within the decellularized ECM, as evidenced by an absence of red staining. Decellularized tissue that was not treated with lipase only slightly reduced lipid levels compared to fresh lipoaspirate, even after 1 week of processing.

### In Vitro Characterization and Gelation of Adipose Matrix

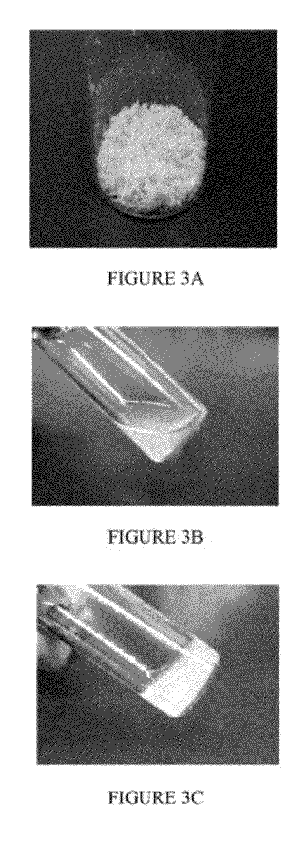

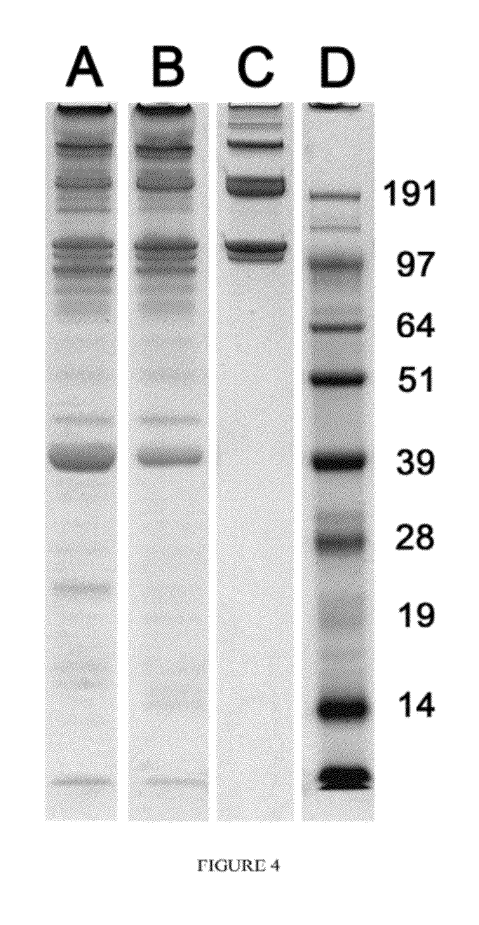

Following decellularization and delipidization, the isolated adipose ECM was lyophilized, milled into a fine powder (FIG. 3A), and then solubilized with pepsin to generate a liquid injectable form of adipose matrix (FIG. 3B). The presence of lipids in the matrix prevented complete lyophilization and efficient solubilization. Groups that did not employ lipase and colipase during the decellularization process remained oily after lyophilization and could not be milled nor fully solubilized, resulting in a highly particulate digest that could not be pushed through a 25 G needle. These groups also exhibited inconsistent gelation in vitro and in vivo. However, groups that were delipidized produced a dry matrix following lyophilization that could be easily milled into a fine powder. SDS-PAGE analysis of digested adipose matrix revealed multiple peptides and low molecular weight peptide fragments. Peptide bands characteristic of collagen were present within the digest, in addition to multiple peptides below 39 kDa (FIG. 4).

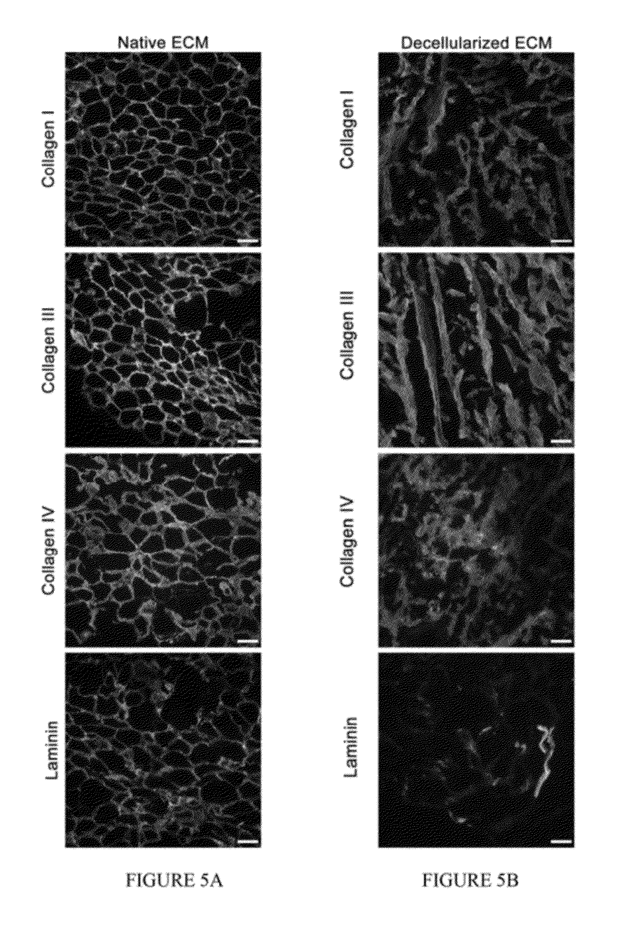

Specifically, collagens I, III, and IV were all present in immunofluorescent stains of adipose tissue both before and after processing (FIG. 5). Collagens I and III were more prevalent, however this could be the result of cross-reactivity of the antibody between isoforms. Laminin was also expressed at both time points, however to a lesser extent after decellularization (FIG. 5). Control slides showed negligible background staining when primary or secondary antibodies were omitted (not shown). Glycosaminoglycan analysis estimated an average of 2.18±0.32 μg of sulfated GAG per mg dry adipose ECM, with no significant difference between tissue decellularized with SDS versus sodium deoxycholate.

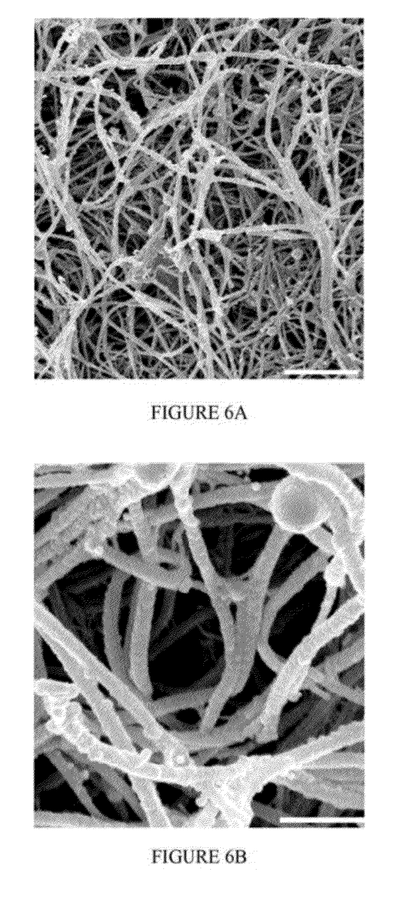

Upon adjusting the pH and temperature of the liquid adipose matrix to physiologic conditions (pH 7.4, 37° C.), the solution self-assembled into a gel (FIG. 3C). SEM analysis revealed the gels were nanofibrous scaffolds with an average fiber diameter of 100 nm and interconnecting pores (FIG. 6). Storage moduli were determined at 1 rads and ranged from 5-9 Pa for tissue processed with SDS and from 7-18 Pa for tissue processed with sodium deoxycholate.

Adipose Matrix Coatings Support hASC Culture In Vitro

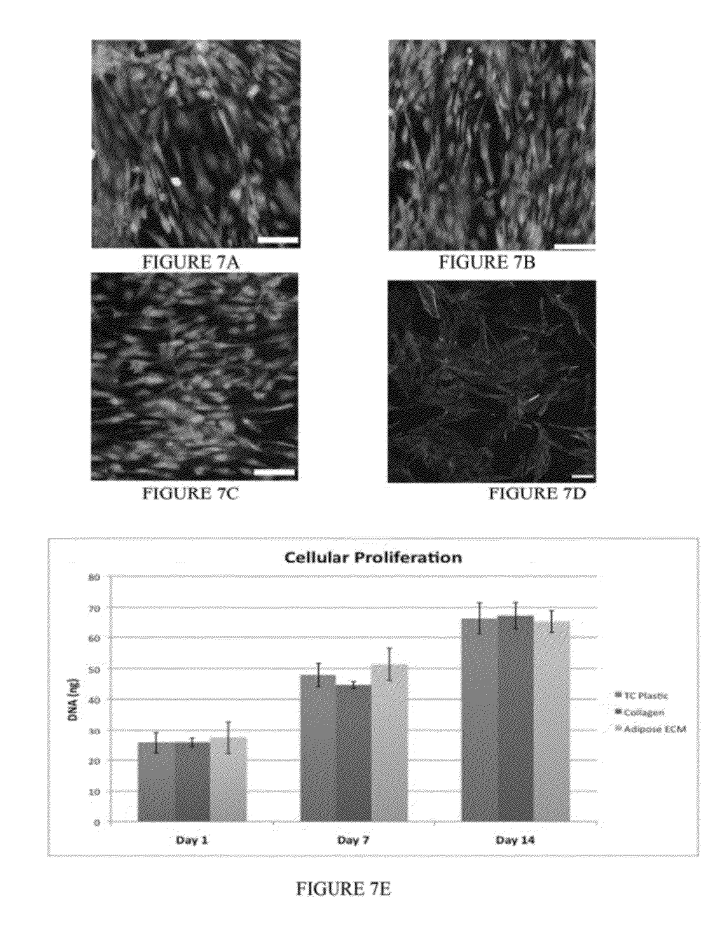

To investigate the ability of the adipose matrix to support cell adhesion and survival, patient-matched hASCs were cultured either on adipose matrix coated tissue culture plates or collagen coated plates, and maintained in growth media. On adipose matrix coated plates, hASCs readily adhered to the surface, displaying a healthy, fibroblast-like phenotype within 24 hours (FIG. 7) [41, 42]. Live/Dead staining revealed negligible cell death on the adipose ECM after 14 days (FIG. 7A-C). This level of viability was consistent regardless of the surface coating. Furthermore, DNA quantification indicated that cellular growth was not hindered by the adipose ECM (FIG. 7E). hASC proliferation continued for 2 weeks on the adipose ECM and was not significantly different from normal proliferation on uncoated or collagen coated surfaces.

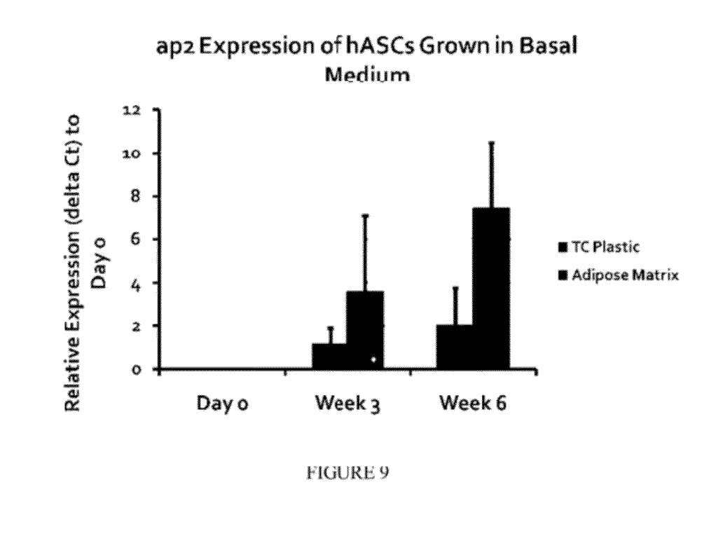

Separately, hASCs were cultured on either tissue culture plastic or adsorbed adipose matrix coating to investigate the adipogenic potential of the adipose matrix. After 6 weeks in static culture with only Growth Medium, expression of fatty acid biding protein (aP2), a later marker of adipogenesis, was upregulated in hASCs cultured on adsorbed adipose matrix coating (FIG. 9). hASCs cultured on standard tissue culture plastic showed negligible expression of aP2 over the 6 weeks, and had significantly lower expression at week 6 compared to hASCs cultured on adipose matrix. These findings suggest that, in the absence of chemical or mechanical differentiation stimuli, the adipose matrix alone encouraged hASCs to proceed towards an adipocyte lineage. Thus, by closely mimicking the natural chemical complexity of adipose tissue, the adipose matrix could provide a signal to encourage maturation of hASCs toward an adipogenic phenotype. This could be particularly advantageous both for studying natural adipogenesis of cells in vitro, or for promoting natural adipose regeneration when the adipose matrix is used as a tissue engineering therapy.

### Gelation of Adipose Matrix In Vivo

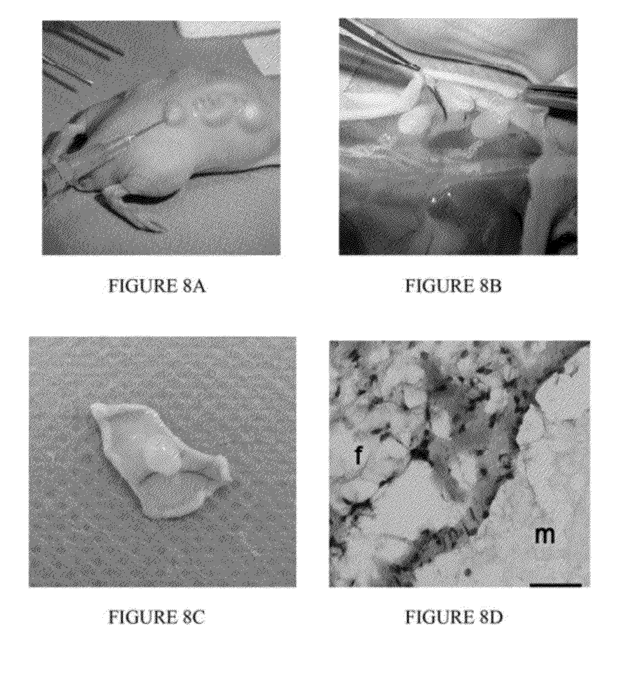

Liquid adipose matrix was injected subcutaneously in mice to investigate in vivo self-assembly (FIG. 8A). Solubilized adipose matrix formed a compact, white bolus when injected subcutaneously using a 25G needle (FIG. 8B). Within 15 minutes, the bolus had solidified into gel that maintained its shape when excised (FIG. 8C). Immediately following injection, the bolus could be pinched or molded to create elongated structures prior to gelation. H&E analysis of excised tissue showed an acellular, porous matrix in close contact with subcutaneous adipose tissue (FIG. 8D).

### Discussion

While several three dimensional scaffolds have been proposed for adipose tissue regeneration, injectable fillers offer unique characteristics that are specifically advantageous for application in adipose tissue. Because adipose regeneration is typically associated with enhancement or contouring of natural features to improve aesthetics, the minimally-invasive delivery of an injectable material is desirable to reduce scarring at the surgical site. Furthermore, the collection of source material from liposuction, as opposed to surgical excision of whole fat pads, compliments this minimally-invasive approach by limiting donor site damage. Injectable materials also allow for contouring of complex features within the face, a common area of desired adipose regeneration. Solid scaffolds cannot offer this level of customization. Consequently, an improved scaffold for adipose tissue engineering would allow for injectable delivery, match the chemical complexity of the native microenvironment, and promote natural regeneration of the tissue as it is resorbed.

Provided herewith is a production of decellularized and delipidized adipose ECM from human lipoaspirate using a combined detergent and enzymatic method. The results presented herewith indicate that decellularized and delipidized lipoaspirate retains a complex composition of proteins, peptides, and glycosaminoglycans (GAGs). Immunofluorescent staining indicated the preservation of multiple collagen isoforms, a major component of native adipose ECM. Despite a slight reduction in content compared to native tissue, laminin was also expressed within the decellularized adipose ECM.

Adipose ECM has been previously reported to contain many of the components of basement membrane, including collagens I, IV, and VI, laminin, and fibronectin [43, 44]. Excessive oils within the lipoaspirate prevented accurate calculation of the GAG content of native adipose tissue using a Blyscan assay. However, there are reports of multiple GAGs and proteoglycans present in the secretome of mouse 3T3-L1 adipocytes, such as perlecan, mimecan, and decorin [43, 45, 46]. It is found native GAGs retained within the adipose matrix material. Currently, a wide range of values have been reported in literature for GAGs retained within solubilized versions of decellularized tissues. Singelyn et al. reported 23.2±4.63 μg GAG per mg solubilized myocardial ECM, but Stern et al. were unable to detect any GAGs within their solubilized skeletal muscle ECM [36, 47].

Clearly there exists extensive variability in ECM composition among tissue types and decellularization protocols. While this decellularization protocol likely causes a reduction in protein and GAG concentration compared to native tissue, this assortment of native biochemical cues mimics the microenvironment of adipose tissue, unlike existing soft-tissue fillers, and can provide adipose specific cues for cell migration, survival, and differentiation. Sulfated GAGs are recognized for their ability to sequester growth factors and subsequently present them to cells [48-50], and thus their presence within the matrix provides an avenue for bioactive molecule delivery both in vitro and in vivo. In addition, PAGE analysis of the injectable adipose matrix confirmed the presence of peptides with a molecular weight at 16 kDa and below, which have been previously shown with other decellularized matrices to have chemoattractant potential [19].

SDS and sodium deoxycholate were used to decellularize the lipoaspirate as they have previously been shown to effectively decellularize multiple tissues [17]. When applied to fresh tissue, these ionic detergents disrupt the cell and nuclear membranes and entrap the freed nuclear contents into micelles, which are then washed away [17, 51]. Through gross and histological observation, it appeared that both SDS and sodium deoxycholate adequately removed all cellular debris. However, by quantifying the extent of decellularization with DNEasy, SDS proved to have a significantly lower amount of contaminating DNA. As to level of DNA is preferred to decellularization. Gilbert et al. suggest that there may exist a threshold DNA concentration below which no immune response will be triggered [52]. It is possible that the detergents also degrade the structure of DNA and other nuclear proteins to an extent that they are no longer recognized as foreign antigens. In fact, many commercially available acellular matrices have been found to contain some degree of cellular contaminants despite their successful use in clinical treatment [52]. Apart from decellularization efficiency, the two detergents appeared to perform at a similar degree. They both produced similar gel electrophoresis bands and GAG content, indicating that neither detergent had a more pronounced deleterious effect on the ECM. Both methods also produced gels that showed a similar range of storage moduli, which align with previously published reports for the modulus of self-assembling collagen gels [53, 54].

Adipose tissue was adept at trapping lipids within its ECM, resulting in multiple complications during processing into an injectable scaffold. While detergents could sufficiently eliminate free lipids surrounding the tissue, a large proportion of oily residue remained trapped on and within the adipose matrix. These sequestered lipids inhibited consistent lyophilization, milling, and solubilization of the adipose matrix. To eliminate lipids from the decellularized adipose matrix, a method inspired by the body's natural lipid metabolism mechanism [55] was produced. Lipase is a naturally occurring esterase produced in the pancreas to digest dietary fats within the small intestine. It specifically targets the ester bond of triglycerides, separating the compound into glycerol and fatty acids, which are readily emulsified by bile salts, such as sodium deoxycholate [56]. Lipase is also actively involved in the breakdown of triglycerides from adipose stores for energy homeostasis [57]. SDS has, however, been shown to cooperatively bind with lipase and irreversibly inhibit its activity [58]. This finding was confirmed in the research and necessitated that sodium deoxycholate be used during lipase digestion, regardless of the initial decellularization detergent (data not shown). Additionally, Labourdenne et al. demonstrated that bile salts can partially inhibit lipase activity, but this inhibition can be overcome by the addition of colipase [59]. They reported that colipase increased lipase activity by 10-15 fold.

Here, it is found that exposing the adipose matrix to lipase in excess of 72 hours resulted in significant protein degradation and an inability to self-assemble following solubilization (data not shown). For this reason, colipase was incorporated to keep enzymatic digestion times to a minimum.

Detergent-based decellularization methods have received criticism for their potential to degrade the extracellular matrix during processing. To avoid the use of detergents, several groups have investigated the direct injection of lipoaspirate via “lipotransfer” operations or the injection of homogenized lipoaspirate emulsifications [12-14, 16, 60]. However, none of these studies attempted to remove cells or lipids from the injected material. While autologous lipid injection should not initiate a foreign antigen response initially, apoptotic cells within the implant could serve as nucleation sites for calcification [61]. Implant calcification has also been associated with the presence of cell membrane phospholipids [62]. Additionally, emulsions of lipids or cellular contents would create heterogeneity within an injectable material, yielding unpredictable material behavior in vivo and limited contouring capability. The sequelae of cellular and lipid remnants in an injected soft tissue filler argue in favor of decellularization despite the possible degradation of proteins. The results presented herewith indicate that decellularized adipose matrix retains much of the protein complexity of native tissue alongside the complete removal of lipids from the material. This removal of both cellular and lipid content reduces concerns surrounding implant immune rejection and calcification.

The results presented herewith demonstrates that human lipoaspirate can be effectively decellularized, delipidized, and subsequently solubilized to produce a self-assembling subcutaneous filler. While not every component of native adipose ECM was fully retained, this adipose matrix is comprised of a complex arrangement of natural proteins and polysaccharides that more closely mimics the in vivo microenvironment than currently approved fillers such as collagen and hyaluronic acid. Furthermore, this material could be used as a delivery vehicle for incorporating adipose derived stem cells in a regenerative treatment. It has been postulated that the success of lipotransfer treatments can be attributed to the presence of a small population of resident hASCs within the injected material [1,3]. Using solubilized adipose matrix as a delivery vehicle, these cells could be delivered in a concentrated and more consistent manner.

Patient-matched hASCs readily proliferated on 2D adipose matrix coatings and showed positive viability. These systems could allow for the investigation of the influence of multiple physical and biochemical parameters on hASC differentiation. Several groups have reported control over adipogenesis using various chemical additives and paracrine signals [63-65]. However, there has been growing literature indicating that the surrounding microenvironment has a significant impact on stem cell fate as well. Here, the invention demonstrates the ability for generating a scaffold derived from human lipoaspirate. Decellularized and delipidized adipose matrix can provide the biochemical cues seen by hASCs in vivo, yet allow the specific control over extraneous conditions offered by an in vitro setting. Thus, this material can be used for both an injectable scaffold for adipose tissue engineering, and a platform for discovering the controlling mechanisms behind adipogenesis.

In summary, the present invention demonstrates the feasibility of human lipoaspirate as a minimally-invasive option for adipose tissue engineering, from collection of source material to delivery of the scaffold. Liposuctioned fat has been collected, processed into an acellular material, digested, and neutralized. This neutralized solution has been shown in the lab to self-assemble into a gel both in the incubator or when injected subcutaneously into the back of female Sprague-Dawley rats. Adipogenic efficiency of the present adipose extracellular matrix in athymic mice is also determined.

While other injectable soft tissue fillers have been investigated, acellular adipose matrix provides a closer approximation to the biochemical compositional complexity of native adipose ECM. The removal of both lipids and cellular contents produces an implant with limited immune concerns, even if the lipoaspirate originates from an allogeneic source. Its gelation at body temperature permits small needle delivery, which would facilitate fine contouring of complex voids. Thus, decellularized and delipidized lipoaspirate produces a potentially autologous soft tissue filler capable of thermally-responsive gelation and minimally-invasive delivery.

Therefore, the present invention provides a tissue specific decellularized and delipidized extracellular matrix derived from adipose or loose connective tissue that retains properties important for the migration and infiltration of native cell types. A better scaffold than many materials currently used as fillers is also provided because of its ability to integrate with existing tissue. A better environment for cell growth is also provided. The adipose extracellular matrix can include the addition of growth factors to the binding receptors in the matrix, which should enhance tissue formation. The adipose extracellular matrix can also be used autologously (via liposuction) to provide an individualized matrix, and can be combined with other materials and various small molecules for specific applications such as skin grafts or certain traumatic injury repair.

The decellularized and delipidized adipose or loose connective tissue extracellular matrix provided by the present invention can be used for a number of applications where new, functional adipose tissue is desired. For instance, the adipose-specific extracellular matrix of the present invention can be especially useful in a number of facial cosmetic surgeries, such as chin, cheek, or forehead lifts. Based on the angiogenic potential of the material, the adipose-specific extracellular matrix can also be used for larger surgeries such as breast or buttock augmentations. Additionally, the adipose-specific extracellular matrix can be used in the treatment of third degree burns to eliminate divots commonly present under large skin grafts. Other surgeries, such as those to repair cleft lip, facial abnormalities, or traumatic injuries to subcutaneous layers, can also make use of the present invention.

### REFERENCES

1. Patrick Jr C, Chauvin P, Hobley J, Reece G. Preadipocyte seeded PLGA scaffolds for adipose tissue engineering. Tissue Engineering. 1999; 5:139-51.
2. von Heimburg D, Zachariah S, Kühling H, Heschel I, Schoof H, Hafemann B, et al. Human preadipocytes seeded on freeze-dried collagen scaffolds investigated in vitro and in vivo. Biomaterials. 2001; 22:429-38.
3. Stillaert F B, Di Bartolo C, Hunt J A, Rhodes N P, Tognana E, Monstrey S, et al. Human clinical experience with adipose precursor cells seeded on hyaluronic acid-based spongy scaffolds. Biomaterials. 2008; 29:3953-9.
4. Marler J J, Guha A, Rowley J, Koka R, Mooney D, Upton J, et al. Soft-tissue augmentation with injectable alginate and syngeneic fibroblasts. Plastic and reconstructive surgery. 0.2000; 105:2049-58.
5. Torio-Padron N, Baerlecken N, Momeni A, Stark G B, Borges J. Engineering of adipose tissue by injection of human preadipocytes in fibrin. Aesthetic plastic surgery. 2007; 31:285-93.
6. Halberstadt C, Austin C, Rowley J, Culberson C, Loebsack A, Wyatt S, et al. A hydrogel material for plastic and reconstructive applications injected into the subcutaneous space of a sheep. Tissue Engineering. 2002; 8:309-19.
7. Hoffmann C, Schuller-Petrovic S, Soyer H P, Kerl H. Adverse reactions after cosmetic lip augmentation with permanent biologically inert implant materials. J Am Acad Dermatol. 1999; 40:100-2.
8. Lemperle G, Morhenn V, Charrier U. Human histology and persistence of various injectable filler substances for soft tissue augmentation. Aesthetic plastic surgery. 2003; 27:354-66; discussion 67.
9. Okabe K, Yamada Y, Ito K, Kohgo T, Yoshimi R, Ueda M. Injectable soft-tissue augmentation by tissue engineering and regenerative medicine with human mesenchymal stromal cells, platelet-rich plasma and hyaluronic acid scaffolds. Cytotherapy. 2009; 11:307-16.
10. Hemmrich K, Van de Sijpe K, Rhodes N P, Hunt J A, Di Bartolo C, Pallua N, et al. Autologous in vivo adipose tissue engineering in hyaluronan-based gels—a pilot study. J Surg Res. 2008; 144:82-8.
11. Tan H, Ramirez C, Miljkovic N, Li H, Rubin J, Marra K. Thermosensitive injectable hyaluronic acid hydrogel for adipose tissue engineering. Biomaterials. 2009; 30:6844-53.
12. Cohen G, Treherne A. Treatment of facial lipoatrophy via autologous fat transfer. J Drugs Dermatol. 2009; 8:486-9.
13. Meier J, Glasgold R, Glasgold M. Autologous Fat Grafting: Long-term Evidence of Its Efficacy in Midfacial Rejuvenation. Archives of Facial Plastic Surgery. 2009; 11:24.
14. Kanchwala S K, Holloway L, Bucky L P. Reliable soft tissue augmentation: a clinical comparison of injectable soft-tissue fillers for facial-volume augmentation. Annals of plastic surgery. 2005; 55:30-5; discussion 5.
15. Patrick C W. Tissue engineering strategies for adipose tissue repair. Anat Rec. 2001; 263:361-6.
16. Toledo L S, Mauad R. Fat injection: a 20-year revision. Clin Plast Surg. 2006; 33:47-53, vi.
17. Gilbert T W, Sellaro T L, Badylak S F. Decellularization of tissues and organs. Biomaterials. 2006; 27:3675-83.
18. Badylak S F, Freytes D O, Gilbert T W. Extracellular matrix as a biological scaffold material: Structure and function. Acta biomaterialia. 2009; 5:1-13.
19. Li F, Li W, Johnson S, Ingram D, Yoder M, Badylak S. Low-molecular-weight peptides derived from extracellular matrix as chemoattractants for primary endothelial cells. Endothelium. 2004; 11:199-206.
20. Reing J E, Zhang L, Myers-Irvin J, Cordero K E, Freytes D O, Heber-Katz E, et al. Degradation products of extracellular matrix affect cell migration and proliferation. Tissue Engineering Part A. 2009; 15:605-14.
21. Ott H C, Matthiesen T S, Goh S-K, Black L D, Kren S M, Netoff T I, et al. Perfusion-decellularized matrix: using nature's platform to engineer a bioartificial heart. Nat. Med. 2008; 14:213-21.
22. Brennan E P, Tang X-H, Stewart-Akers A M, Gudas L J, Badylak S F. Chemoattractant activity of degradation products of fetal and adult skin extracellular matrix for keratinocyte progenitor cells. J Tissue Eng Regen Med. 2008; 2:491-8.
23. Abberton K M, Bortolotto S K, Woods A A, Findlay M, Morrison W A, Thompson E W, et al. Myogel, a novel, basement membrane-rich, extracellular matrix derived from skeletal muscle, is highly adipogenic in vivo and in vitro. Cells Tissues Organs (Print). 2008; 188:347-58.
24. Flynn L, Semple J L, Woodhouse K A. Decellularized placental matrices for adipose tissue engineering. J Biomed Mater Res. 2006; 79:359-69.
25. Uriel S, Labay E, Francis-Sedlak M, Moya M, Weichselbaum R, Ervin N, et al. Extraction and Assembly of Tissue-Derived Gels for Cell Culture and Tissue Engineering. Tissue engineering Part C, Methods. 2008.
26. Leor J, Amsalem Y, Cohen S. Cells, scaffolds, and molecules for myocardial tissue engineering. Pharmacol Ther. 2005; 105:151-63.
27. Badylak S F. The extracellular matrix as a biologic scaffold material. Biomaterials. 2007; 28:3587-93.
28. Uriel S, Labay E, Francis-Sedlak M, Moya M L, Weichselbaum R R, Ervin N, et al. Extraction and Assembly of Tissue-Derived Gels for Cell Culture and Tissue Engineering. Tissue Eng Part C Methods. 2008.
29. Lutolf M P, Hubbell J A. Synthetic biomaterials as instructive extracellular microenvironments for morphogenesis in tissue engineering. Nat. Biotechnol. 2005; 23:47-55.
30. Macfelda K, Kapeller B, Wilbacher I, Losert U M. Behavior of cardiomyocytes and skeletal muscle cells on different extracellular matrix components—relevance for cardiac tissue engineering. Artif Organs. 2007; 31:4-12.
31. Brown L. Cardiac extracellular matrix: a dynamic entity. Am J Physiol Heart Circ Physiol. 2005; 289:H973-4.
32. Choi J S, Yang H-J, Kim B S, Kim J D, Lee S-H, Lee E K, et al. Fabrication of Porous Extracellular Matrix (ECM) Scaffolds from Human Adipose Tissue. Tissue engineering Part C, Methods. 2009.
33. Flynn L E. The use of decellularized adipose tissue to provide an inductive microenvironment for the adipogenic differentiation of human adipose-derived stem cells. Biomaterials. 2010; 31:4715-24.
34. Zuk P, Zhu M, Mizuno H, Huang J, Futrell J, Katz A, et al. Multilineage cells from human adipose tissue: implications for cell-based therapies. Tissue Engineering. 2001; 7:211-28.
35. Bernacki S, Wall M, Loboa E. Isolation of human mesenchymal stem cells from bone and adipose tissue. Methods in cell biology. 2008; 86:257.
36. Singelyn J M, DeQuach J A, Seif-Naraghi S B, Littlefield R B, Schup-Magoffin P J, Christman K L. Naturally derived myocardial matrix as an injectable scaffold for cardiac tissue engineering. Biomaterials. 2009; 30:5409-16.
37. Hudson T W, Liu S Y, Schmidt C E. Engineering an improved acellular nerve graft via optimized chemical processing. Tissue Engineering. 2004; 10:1346-58.
38. Cheng H-W, Tsui Y-K, Cheung K M C, Chan D, Chan B P. Decellularization of chondrocyte-encapsulated collagen microspheres: a three-dimensional model to study the effects of acellular matrix on stem cell fate. Tissue engineering Part C, Methods. 2009; 15:697-706.
39. Koopman R, Schaart G, Hesselink M K. Optimisation of oil red O staining permits combination with immunofluorescence and automated quantification of lipids. Histochem Cell Biol. 2001; 116:63-8.
40. Freytes D O, Martin J, Velankar S S, Lee A S, Badylak S F. Preparation and rheological characterization of a gel form of the porcine urinary bladder matrix. Biomaterials. 2008; 29:1630-7.
41. Zuk P, Zhu M, Ashjian P, De Ugarte D, Huang J, Mizuno H, et al. Human adipose tissue is a source of multipotent stem cells. Molecular biology of the cell. 2002; 13:4279.
42. Gimble J, Guilak F. Adipose-derived adult stem cells: isolation, characterization, and differentiation potential. Cytotherapy. 2003; 5:362-9.
43. Mariman E C M, Wang P. Adipocyte extracellular matrix composition, dynamics and role in obesity. Cell Mol Life Sci. 2010; 67:1277-92.
44. Wang P, Mariman E, Keijer J, Bouwman F, Noben J-P, Robben J, et al. Profiling of the secreted proteins during 3T3-L1 adipocyte differentiation leads to the identification of novel adipokines. Cell Mol Life Sci. 2004; 61:2405-17.
45. Lim J-M, Sherling D, Teo C F, Hausman D B, Lin D, Wells L. Defining the regulated secreted proteome of rodent adipocytes upon the induction of insulin resistance. J Proteome Res. 2008; 7:1251-63.
46. Roelofsen H, Dijkstra M, Weening D, de Vries M P, Hoek A, Vonk R J. Comparison of isotope-labeled amino acid incorporation rates (CILAIR) provides a quantitative method to study tissue secretomes. Mol Cell Proteomics. 2009; 8:316-24.
47. Stern M M, Myers R L, Hammam N, Stern K A, Eberli D, Kritchevsky S B, et al. The influence of extracellular matrix derived from skeletal muscle tissue on the proliferation and differentiation of myogenic progenitor cells ex vivo. Biomaterials. 2009; 30:2393-9.
48. Mullen L M, Best S M, Brooks R A, Ghose S, Gwynne J H, Wardale J, et al. Binding and Release Characteristics of Insulin-Like Growth Factor-1 from a Collagen-Glycosaminoglycan Scaffold. Tissue engineering Part C, Methods. 2010.
49. Doran M R, Markway B D, Aird I A, Rowlands A S, George P A, Nielsen L K, et al. Surface-bound stem cell factor and the promotion of hematopoietic cell expansion. Biomaterials. 2009; 30:4047-52.
50. Yayon A, Klagsbrun M, Esko J D, Leder P, Ornitz D M. Cell surface, heparin-like molecules are required for binding of basic fibroblast growth factor to its high affinity receptor. Cell. 1991; 64:841-8.
51. Seddon A M, Curnow P, Booth P J. Membrane proteins, lipids and detergents: not just a soap opera. Biochim Biophys Acta. 2004; 1666:105-17.
52. Gilbert T W, Freund J M, Badylak S F. Quantification of DNA in biologic scaffold materials. Surg Res. 2009; 152:135-9.
53. Raub C B, Putnam A J, Tromberg B J, George S C. Predicting bulk mechanical properties of cellularized collagen gels using multiphoton microscopy. Acta biomaterialia. 2010.
54. Miron-Mendoza M, Seemann J, Grinnell F. The differential regulation of cell motile activity through matrix stiffness and porosity in three dimensional collagen matrices. Biomaterials. 2010; 31:6425-35.
55. Van Tilbeurgh H, Bezzine S, Cambillau C, Verger R, Carriere F. Colipase: structure and interaction with pancreatic lipase. Biochim Biophys Acta. 1999; 1441:173-84.
56. Lafontan M, Langin D. Lipolysis and lipid mobilization in human adipose tissue. Prog Lipid Res. 2009; 48:275-97.
57. Zimmermann R, Strauss J G, Haemmerle G, Schoiswohl G, Birner-Gruenberger R, Riederer M, et al. Fat mobilization in adipose tissue is promoted by adipose triglyceride lipase. Science. 2004; 306:1383-6.
58. Borgstrom B, Donner J. Interactions of pancreatic lipase with bile salts and dodecyl sulfate. The Journal of Lipid Research. 1976; 17:491.
59. Labourdenne S, Brass O, Ivanova M, Cagna A, Verger R. Effects of colipase and bile salts on the catalytic activity of human pancreatic lipase. A study using the oil drop tensiometer. Biochemistry. 1997; 36:3423-9.
60. Choi J S, Yang H-J, Kim B S, Kim J D, Kim J Y, Yoo B, et al. Human extracellular matrix (ECM) powders for injectable cell delivery and adipose tissue engineering. J Control Release. 2009; 139:2-7.
61. Proudfoot D, Skepper J N, Hegyi L, Bennett M R, Shanahan C M, Weissberg P L. Apoptosis regulates human vascular calcification in vitro: evidence for initiation of vascular calcification by apoptotic bodies. Circulation Research. 2000; 87:1055-62.
62. Hirsch D, Drader J, Thomas T J, Schoen F J, Levy J T, Levy R J. Inhibition of calcification of glutaraldehyde pretreated porcine aortic valve cusps with sodium dodecyl sulfate: preincubation and controlled release studies. J Biomed Mater Res. 1993; 27:1477-84.
63. Hebert T L, Wu X, Yu G, Goh B C, Halvorsen Y-DC, Wang Z, et al. Culture effects of epidermal growth factor (EGF) and basic fibroblast growth factor (bFGF) on cryopreserved human adipose-derived stromal/stem cell proliferation and adipogenesis. J Tissue Eng Regen Med. 2009; 3:553-61.
64. Hemmrich K, von Heimburg D, Cierpka K, Haydarlioglu S, Pallua N. Optimization of the differentiation of human preadipocytes in vitro. Differentiation. 2005; 73:28-35.
65. van Harmelen V, Skurk T, Hauner H. Primary culture and differentiation of human adipocyte precursor cells. Methods Mol. Med. 2005; 107:125-35.

1. A composition comprising an aqueous solution and a decellularized and delipidized extracellular matrix derived from adipose or loose connective tissue, wherein said decellularized and delipidized extracellular matrix comprises native polypeptides or polysaccharides.

2. The composition of claim 1, wherein the composition comprises native collagens I, III, and IV and laminin.

3. The composition of claim 1, wherein the composition further comprises a digestive enzyme.

4. The composition of claim 3, wherein the enzyme is pepsin.

5. The composition of claim 1, wherein the composition is an injectable thermally responsive hydrogel that is in a liquid form at a temperature below 25° C. and is in a gel form at a temperature greater than 35° C.

6. The composition of claim 1, wherein the composition is formulated to be delivered to a tissue through a 25G or smaller needle.

7. The composition of claim 1, further comprising a natural or synthetic polymer, a growth factor, a chemotaxis factor, a neovascularization factor, an antibiotic agent, an anti-inflammatory agent, or a therapeutic agent.

8. The composition of claim 1, further comprising exogenous cells selected from the group consisting of pluripotent stem cells, multipotent stem cells, progenitor cells, adipose-derived mesenchymal stem cells, adipocytes, or lipoblasts.

9. The composition of claim 1, wherein said adipose or loose connective tissue is obtained from lipoaspirate.

10. The composition of claim 1, wherein said decellularized and delipidized extracellular matrix is formulated to coat a tissue culture device to pluripotent stem cells, multipotent stem cells, progenitor cells, adipose-derived mesenchymal stem cells, adipocytes, or lipoblasts.

11. A method of producing a composition comprising a decellularized and delipidized extracellular matrix derived from adipose or loose connective tissue, comprising:
(a) decellularizing an adipose or loose connective tissue with a detergent agent to obtain decellularized adipose or loose tissue extracellular matrix;
(b) delipidizing the decellularized adipose or loose tissue extracellular matrix with a delipidizing agent to obtain decellularized and delipidized adipose or loose tissue extracellular matrix; and
(c) digesting the decellularized and delipidized adipose or loose connective tissue matrix with a protein or glycosaminoglycan digestive enzyme.

12. The method of claim 1 wherein said detergent agent is selected from sodium dodecyl sulfate (SDS), sodium deoxycholate, and combinations thereof.

13. The method of claim 11, wherein said delipidizing agent is selected from lipase, colipase, and combinations thereof.

14. The method of claim 11, wherein the digesting enzyme is pepsin.

15. The method of claim 11, further comprising an earlier step of obtaining the adipose or loose connective tissue from lipoaspirate.

16. The method of claim 11, further comprising a later step of lyophilizing the decellularized and delipidized extracellular matrix.

17. The method of claim 16, further comprising a later step of suspending and neutralizing the digested decellularized and delipidized extracellular matrix in a water, saline or phosphate buffered solution.

18. The method of claim 17, further comprising a later step of re-lyophilizing the extracellular matrix in a solution and then rehydrating with water, saline or phosphate buffered solution.

19. The method of claim 17, further comprising a later step of coating a tissue culture device with the suspended decellularized and delipidized extracellular matrix.

20. The method of claim 17, wherein said solubilized, decellularized and delipidized extracellular matrix spontaneously forms into a gel at above 35° C.

21. A method of providing to an individual an adipose matrix scaffold comprising parenterally administering to or implanting into an individual in need thereof an effective amount of the composition of claim 17.

22. The method of claim 21, wherein said composition further comprises exogenous cells, natural or synthetic polymers, growth factors, antibiotic agents, neovascularization agents, anti-inflammatory agents, or therapeutic agents.

23. A method of culturing cells on an adsorbed matrix comprising the steps of:
(a) providing a composition comprising an aqueous solution and a decellularized, delipidized, and enzymatically digested extracellular matrix derived from adipose or loose connective tissue into a tissue culture device;
(b) incubating said tissue culture device to adsorb at least some of the decellularized and delipidized extracellular matrix onto the device; and
(c) culturing cells on the adsorbed matrix.

24. The method of claim 21, wherein said cells are selected from the group consisting of pluripotent stem cells, multipotent stem cells, progenitor cells, adipose-derived mesenchymal stem cells, adipocytes, or lipoblasts.

25. The method of claim 23, wherein the adipose or loose connective tissue is obtained from lipoaspirate.
